(19) United States
(12) Patent Application Publication
(10) Pub. No.: US 2007/0275925 A1
(43) Pub. Date: Nov. 29, 2007

(54) ASTHMA DIAGNOSIS AND THERAPY

(75) Inventors: Prescott G. Woodruff, San Francisco, CA (US); John V. Fahy, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA

(21) Appl. No.: 11/748,375

(22) Filed: May 14, 2007

Related U.S. Application Data
(60) Provisional application No. 60/801,289, filed on May 17, 2006.

(51) Int. Cl. A61K 48/00; C12Q 1/68

(52) U.S. Cl. 514/44; 435/6

(57) ABSTRACT

The invention provides drug assays, methods of diagnosing, and methods and compositions for treating asthma and other lung disease based upon the identification and/or use of agents which modulate CST1, HDAC9 or PRR4 levels or activity.

### CROSS-REFERENCES TO RELATED APPLICATIONS

The present application claims priority to U.S. Ser. No. 60/801,289, filed May 17, 2006, herein incorporated by reference in its entirety.

### STATEMENT AS TO RIGHTS TO INVENTIONS MADE UNDER FEDERALLY SPONSORED RESEARCH AND DEVELOPMENT

Supported by grants from NIH(HL72301, HL56385, HL66564, RR17002 and HL072915). The U.S. government may have certain rights in the invention.

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Although multiple epithelial cell abnormalities have been described in asthma, including dysregulation of mucin genes, chemokines, and growth factors, and have led to hypotheses about mechanism, it likely that additional mechanisms of airway epithelial cell dysfunction in asthma remain. A powerful unbiased strategy to uncover unsuspected mechanisms of disease is the application of genome-wide profiling using high-density microarrays.¹ Ohtani et al. used such methods in comparing gene expression profiles in apparently normal, unstimulated respiratory epithelial cells to apparently normal, respiratory epithelial cells stimulated to differentiate into goblet cells by IL13 in vitro. Ohtani et al. reported on many hundreds of genes which were differentially expressed and could be candidates for further evaluation as possible markers for asthma and chronic obstructive pulmonary disease (see, U.S. Patent Application Publication No. 20050208496). However, in the absence of sufficiently conservative statistical analyses to overcome spurious findings associated with the great many comparisons made, there is uncertainty as to the statistical validity of any single finding. Indeed, when Laprise et al. (see, Laprise et al, BMC Genomics 5:21 (2004) compared the gene expression profiles of asthmatic and non-asthmatic subjects a much smaller set of 79 differentially expressed genes were identified and of these they identified a much smaller subset of 26 genes which were partially or completed corrected in the asthmatics by inhaled corticosteroid therapy. The Laprise et al. methods did not include a control for alternative causes of lung inflammation than asthma and thus may not well distinguish pathological mechanism unique to asthma as opposed to pulmonary inflammation.

The goal of this study was to comprehensively characterize the gene expression changes in the airway epithelial compartment in asthma to uncover unsuspected mechanisms of disease. In order to achieve our goal, we sampled the airway epithelium using bronchoscopy and cytology brushes, and incorporated two elements designed establish whether the observed gene expression changes are specific for asthma and whether they play a mechanistic role in the disease. These elements were: 1) the inclusion of a two control groups (non-smoking healthy controls and non-asthmatic smokers with mild/moderate airflow obstruction), and 2) the enrollment of a subgroup of asthmatics in a randomized double-blind placebo-controlled trial of treatment with inhaled corticosteroids. Asthma, unlike COPD and most other airway diseases, is very responsive to inhaled corticosteroids.^{2,3} We reasoned that this characteristic of asthma could be leveraged to help identify genes mechanistically involved in the pathophysiology of asthma and in its treatment response. In addition, for certain genes expression was examined using a taqman assay. Here, we report on the discovery of a much more focused set of genes which are specifically implicated in the etiology and treatment of asthma. Accordingly, these genes and their gene products are targets for the identification and use of new agents in asthma therapy, as well as, allergic rhinitis, chronic obstructive pulmonary disease, or inflammatory or fibrotic diseases of the lung.

### BRIEF SUMMARY

The invention relates to the discovery of PRR4, HDAC9, and CST1 as novel mediators of asthma, and further that epithelial expression of HDAC9, PRR4, and CST1 predicts response (or lack thereof in the case of PRR4) to corticosteroids in asthmatics. Accordingly, in a first aspect, the invention provides methods of identifying agents useful in the treatment of asthma, allergic rhinitis, chronic obstructive pulmonary disease, or inflammatory or fibrotic diseases of the lung said method comprising determining the ability of the agent modulate PRR4, HDAC9, and CST1 levels or activity. In this aspect, the invention also provides methods and compositions useful in treating asthma, allergic rhinitis, chronic obstructive pulmonary disease, or inflammatory or fibrotic diseases of the lung which comprise agents identified for use in treating asthma according to their ability to modulate PRR4, HDAC9, and CST1 levels or activity. In another aspect, the invention provides methods for treating asthma, allergic rhinitis, chronic obstructive pulmonary disease, or inflammatory or fibrotic diseases of the lung which involve the administration of a modulator of CST1, HDAC9 or PRR4 levels or activity. In yet a further aspect, the invention provides pharmaceutical compositions comprising modulators of PRR4, HDAC9, and CST1 levels or activity which can be used in treating asthma, allergic rhinitis, chronic obstructive pulmonary disease, or inflammatory or fibrotic diseases of the lung. In another aspect, PRR4 is used as a target to identify drugs useful in treating steroid resistant asthma.

In an additional aspect, the invention provides methods of identifying an individual having an increased susceptibility or resistance to asthma, allergic rhinitis, chronic obstructive pulmonary disease, or inflammatory or fibrotic diseases of the lung, said method comprising determining the levels or activity of PRR4, HDAC9, and CST1 in a tissue sample from the individual. In a further aspect, the invention provides for the use of PRR4, HDAC9, and CST1 levels or activity measurements in the diagnosis or prognosis or assessment of response to therapy of an individual having or suspected of having asthma, allergic rhinitis, chronic obstructive pulmonary disease, or inflammatory or fibrotic diseases of the lung. Additionally, HDAC9 and CST1 levels can be used to predict positive response to steroid treatment, while PRR4 levels can be used to diagnose and to predict a negative response to steroid treatment and to predict steroid resistant asthma.

In another aspect, the invention provides for compositions of isolated PRR4, HDAC9, and CST1 proteins or nucleotides encoding the proteins for use in the testing of agents for their usefulness in treating asthma, allergic rhinitis, chronic obstructive pulmonary disease, or inflammatory or fibrotic diseases of the lung.

In another aspect the invention provides a transgenic mouse which over-expresses PRR4, HDAC9, or CST1 in airway epithelial cells. These mice will be useful in the study of airway remodeling and also of bronchial hyper-responsiveness, including asthma, and particularly with respect to cytokine IL-13 action in asthma, as well as allergic rhinitis, chronic obstructive pulmonary disease, or inflammatory or fibrotic diseases of the lung.

In another aspect, the invention provides reagents, including siRNA, to silence PRR4, HDAC9, and CST1 gene expression in airway epithelial cells. These reagents will also be useful in the study of airway remodeling and also of bronchial hyper-responsiveness, including asthma, allergic rhinitis, chronic obstructive pulmonary disease, or inflammatory or fibrotic diseases of the lung.

In still another aspect, the invention provides methods of predicting response to corticosteroid therapy in asthmatics and subjects with allergic rhinitis, COPD, and inflammatory and fibrotic lung disease by determining the level or activity of HDAC9, PRR4, and CST1 in samples of tissue, including but not limited to, the airway epithelial tissue of asthmatics or subjects with allergic rhinitis, COPD, and inflammatory and fibrotic lung disease. HDAC9 and CST1 levels are used to predict a positive response to steroid treatment, while PRR4 levels predict steroid resistant asthma.

In still another aspect, the invention provides pharmaceutical compositions comprising PRR4, HDAC9, and CST1 modulators for treating inflammatory or obstructive diseases of the respiratory tract, particularly asthma and/or chronic obstructive pulmonary disease (COPD) as well as allergic rhinitis, and inflammatory and fibrotic lung disease. In another aspect, the invention provides methods of treating inflammatory or obstructive diseases of the respiratory tract, particularly asthma and/or chronic obstructive pulmonary disease (COPD) as well as allergic rhinitis, and inflammatory and fibrotic lung disease, by administering the PRR4, HDAC9, and CST1 modulators for use according to the invention. In some further embodiments of the above, the modulators are PRR4, HDAC9, and CST1 inhibitors selected from siRNA, antibodies, small organic molecules, and ribozymes.

In another aspect, the present invention provides a method of diagnosing asthma, allergic rhinitis, chronic obstructive pulmonary disease, or inflammatory or fibrotic diseases of the lung in a subject, the method comprising the steps of: (a) contacting a sample from the subject with a reagent that specifically binds to PRR4, HDAC9, or CST1 protein or nucleic acid; and (b) determining the level of PRR4, HDAC9, or CST1 protein or nucleic acid expression in the sample as compared to a control sample, thereby diagnosing asthma, allergic rhinitis, chronic obstructive pulmonary disease, or inflammatory or fibrotic diseases of the lung in a subject.

### BRIEF DESCRIPTION OF THE DRAWINGS

FIG. 1: Schema of the double-blind randomized placebo controlled trial of inhaled corticosteroids. Bronchoscopy was performed before and one week after randomization (a); lung function was measured at baseline and after 4 and 8 weeks of therapy (b). Lung function is presented as FEV₁ which denotes the volume of air exhaled in the first second of a forced expiratory maneuver (in liters). * p=0.028, † p=0.014, ‡ p=0.057 as compared to the change observed in placebo group. Twenty nine of 32 subjects completed the study through the fourth week of study treatment, and 27 completed the eight week of treatment.

FIG. 2: PCR validation of selected genes differentially expressed in epithelial brushings from asthmatic subjects as compared to healthy control subjects (a) and genes responsive to corticosteroids in the clinical trial of inhaled fluticasone in asthmatics (b). Fold induction by microarray was statistically significant (p<0.05, Bonferroni corrected) for all genes shown. Fold induction by PCR (for validation) was statistically significant (p<0.05) for all genes except periostin (POSTN, p=0.064).

FIG. 3: Although there were within group improvements in PC₂₀ to methacholine in the fluticasone treated arm (p<0.05 at 4 and 8 weeks) in the clinical trial, there are no statistically significant differences as compared to placebo.

### DETAILED DESCRIPTION

Introduction

This invention provides methods and compositions useful in studying the pathophysiology of asthma, COPD, allergic rhinitis, and inflammatory and fibrotic lung disease, methods and compositions useful in the treatment of asthma, COPD, allergic rhinitis, and inflammatory and fibrotic lung disease, methods and compositions useful in the identification and risk characterization of individuals having or at risk of having asthma, COPD, allergic rhinitis, and inflammatory and fibrotic lung disease, and methods and compositions useful in identifying modulators useful in the treatment of asthma, COPD, allergic rhinitis, and inflammatory and fibrotic lung disease.

In a first aspect, the invention provides methods of identifying agents useful in the treatment of asthma, COPD, allergic rhinitis, and inflammatory and fibrotic lung disease by determining the ability of the agent to modulate PRR4, HDAC9, and CST1 levels or activity. In some embodiments, this ability is determined by measuring PRR4, HDAC9, or CST1 levels or activity in an experimental sample contacted with the agent and in a reference or control sample which was not contacted with the agent; and comparing the measurements of the experimental and reference samples. In some embodiments, the sample comprises a sample of epithelial tissue, preferably airway epithelium including, but not limited to, airway epithelium obtained from an asthmatic individual or an individual with allergic rhinitis, COPD, or inflammatory or fibrotic lung disease. In some embodiments, the agent is siRNA, an oligonucleotide, a small organic molecule, a ribozyme, a polypeptide, or an antibody. In yet other embodiments, the level of a polynucleotide which encodes PRR4, HDAC9, or CST1 is determined. In still other embodiments, the level of PRR4, HDAC9, or CST1 protein in a cell is measured.

In other embodiments, the ability of an agent to inhibit PRR4, HDAC9, or CST1 is determined by contacting the agent with PRR4, HDAC9, or CST1 and the binding of the agent to the PRR4, HDAC9, or CST1 is determined. In further embodiments, the reference and experimental sample are samples of epithelial tissue. In preferred embodiments, the tissue is airway epithelium, including, but not limited to, tissue from an asthmatic individual. In some embodiments, the sample contains or consists essentially of an isolated PRR4, HDAC9, or CST1 protein. In further embodiments of any of the above, the activity of one or more of the markers is determined. In other embodiments of the above, the agent is a small organic molecule, a polypeptide, or an antibody specific for PRR4, HDAC9, or CST1. Antibodies include monoclonal and polyclonal antibodies. Where the agent is an antibody, it is preferably a monoclonal antibody. More preferably, the antibody is human monoclonal antibody or a humanized monoclonal antibody which is specific for PRR4, HDAC9, or CST1.

In another other aspect, the invention provides methods of treating asthma, COPD, allergic rhinitis, and inflammatory and fibrotic lung disease, by administering an agent which is a modulator of PRR4, HDAC9, or CST1 levels or activity identified or first identified according to any method as described above. In this aspect, the invention further provides pharmaceutical compositions comprising the agent and a pharmacologically acceptable excipient or carrier. Although other routes of administration are suitable, preferably, the above compositions and medicaments are formulated for administration via inhalation.

In still a further aspect, the invention provides methods of treating asthma, COPD, allergic rhinitis, and inflammatory and fibrotic lung disease, comprising administration of a modulator of PRR4, HDAC9, or CST1 levels or activity. In this aspect, the invention further provides pharmaceutical compositions comprising the agent and a pharmacologically acceptable excipient or carrier. Although other routes of administration are suitable, preferably, the above compositions and medicaments are formulated for administration via inhalation. In some embodiments, the treatments reduce the frequency, severity, or recurrence of asthmatic attacks, reduce shortness of breath, cough, or wheezing, improve tissue oxygenation, decrease resistance to air flow in the respiratory tract, increase vital capacity, forced expiratory air volume, peak expiratory air flows, reduce collagen deposition in the respiratory tract, reduce vascular remodeling in the respiratory tract, or smooth muscle proliferation in the respiratory tract in subjects having asthma, COPD, allergic rhinitis, and inflammatory and fibrotic lung disease.

In the next aspect, the invention provides methods of diagnosis and prognosis for individual having and increased susceptibility or resistance to asthma, COPD, allergic rhinitis, and inflammatory and fibrotic lung disease In these methods, the baseline or current levels or activity of PRR4, HDAC9, or CST1 in a tissue sample from the individual is determined and compared to a reference level from a comparison population (e.g., a non-asthmatic or lung-diseased population, an asthmatic or lung diseased subject with controlled disease, or an asthmatic who is responsive or non-response to corticosteroid therapy). The levels and activity information is used to determine the likelihood of an individual experiencing an asthmatic attack or disease occurrence, the possible severity of such attacks, and/or the responsiveness of the individual to therapy with anti-asthma and anti-lung disease drugs, including corticosteroids and other agents. In preferred embodiments, the tissue samples comprises epithelial cells from the patient, preferably, the samples are of airway bronchi, or lung. In some embodiments, PRR4, HDAC9, or CST1 nucleotide levels are measured. In other embodiments PRR4, HDAC9, or CST1 protein levels are measured. In still other embodiments, the activity of PRR4, HDAC9, or CST1 activity levels are measured.

In another aspect the invention provides a transgenic mouse which over-expresses PRR4, HDAC9, or CST1 in airway epithelial cells. These mice will be useful in the study of airway remodeling and also of bronchial hyper-responsiveness, including asthma, COPD, allergic rhinitis, and inflammatory and fibrotic lung disease, and particularly with respect to cytokine IL-13 action in asthma.

In another aspect, the invention provides reagents, including siRNA, to silence PRR4, HDAC9, or CST1 gene expression in airway epithelial cells. These reagents will also be useful in the study of airway remodeling and also of bronchial hyper-responsiveness, including asthma, COPD, allergic rhinitis, and inflammatory and fibrotic lung disease.

Accordingly, the invention also provides a pharmaceutical composition comprising an PRR4, HDAC9, or CST1 siRNA molecule and/or an siRNA expression vector. In further embodiments, the siRNA is at least about 15-50 nucleotides in length (e.g., each complementary sequence of the double stranded siRNA is 15-50 nucleotides in length, and the double stranded siRNA is about 15-50 base pairs in length). In some further embodiments still, the length of the siRNA molecule is about 20-30 base nucleotides, about 20-25 or about 24-29 nucleotides in length, e.g., 20, 21, 22, 23, 24, 25, 26, 27, 28, 29, or 30 nucleotides in length. In still further embodiments, the siRNA is a hairpin loop siRNA.

Accordingly, the invention also provides pharmaceutical compositions comprising an ribozyme which is capable of inhibiting the expression of PRR4, HDAC9, or CST1 in a host cell transduced with the ribozyme.

In some embodiments, modulators of PRR4, HDAC9, or CST1 are used to study the pathophysiology of asthma, COPD, allergic rhinitis, and inflammatory and fibrotic lung disease.

HDAC9 is a member of the Class II histone deacetylases (HDAC4, HDAC5, HDAC7 and HDAC9)— a gene family which function as signal-responsive transcriptional co-repressors and as regulators of MHCII expression. Here we show upregulation in asthmatic epithelial cells and sensitivity to steroids. There are multiple possible mechanism by which HDAC9 could contribute to abnormal cell signaling in asthmatic epithelial cells and our data suggest that this protein represents a previously unsuspected therapeutic target in asthma.

PRR4: aka lacrimal proline-rich protein (LPRP) has been mainly described as a transcript found in lacrimal glands and salivary glands. The importance of our finding is that it PRR4 gene expression is increased in asthmatic airway epithelial cells and that the expression was only modestly reduced by fluticasone. Thus, finding ways to suppress expression of PRR4 may prove to have additive benefit to steroids in asthma or to provide particular therapeutic benefit to asthmatics who are steroid resistant.

CST1 is a protease inhibitor. Proteases and proteases inhibitors are candidate mediators of airway inflammation and remodeling.

Underlying Discoveries Related to the Invention

In genome wide profiling studies of airway epithelial cells in asthma we have discovered that PRR4 and CST1 gene expression are significantly upregulated and their expression is inversely associated with measures of airflow obstruction. In addition, taqman assays show that HDAC9 and CST1 are upregulated in asthma. Taqman assays also show that HDAC9 is responsive to steroids. In additional gene profiling experiments, we found that CST1 gene expression was upregulated in airway epithelial brushing from asthmatics treatment with inhaled fluticasone. In these subjects the baseline expression pf CST1 in airway epithelial brushings predicted responsiveness to corticosteroids. PRR4 levels respond only modestly to steroid treatment, and so PRR4 is useful as a target for identifying drugs to treat asthma and steroid resistant asthma. In addition, PRR4 levels are useful as a diagnostic tool to identify asthma and steroid resistant asthma. HDAC9 and CST1 are useful targets for identifying asthma drugs. In addition, HDAC9 and CST1 levels are useful for diagnosing asthma and steroid responsive asthma.

Without being limited to one theory, the likely role of some of these genes is to promote airway remodeling. The more remodeled the airway lumen, the more airflow obstruction there will be. Thus, expression levels in blood will be highest in asthmatics with the most airflow obstruction. PRR4, HDAC9, or CST are useful as a diagnostic test for asthma, as well as a test of asthma severity. Such diagnostic assays using blood samples would simplify testing and avoid the need for lung function testing.

High-density microarrays are powerful tools for discovery and their application in disease may uncover unsuspected mechanisms and provide new markers of treatment response. Asthma is a common disease with persistent questions about mechanisms and unmet needs in terms of treatment and predictors of treatment response. Allergic inflammation and subsequent remodeling of the airway epithelium occurs in asthma and contributes to the pathophysiology of airflow obstruction and mucus hypersecretion. We used microarrays providing genome-wide coverage to analyze gene expression in airway epithelial cells in asthma and to explore the relationship between epithelial gene expression and airflow response to anti-inflammatory treatment.

Using this approach, we found differential expression of genes in airway epithelial cells previously unassociated with asthma that represent candidate mediators of airway remodeling, and we found genes that predict improvement in airflow with corticosteroid treatment.

Using a very conservative statistical approach, we found 22 genes differentially expressed in epithelial cells from asthma, which, with one exception, were distinct from 40 genes differentially expressed in habitual smokers. We recognize that a less stringent analysis might yield additional information through the identification of a greater number of disease-associated genes. However, we chose a conservative approach to maximize the likelihood that genes identified by microarray analysis would be confirmed by PCR and protein analyses. Three of the 22 genes differentially expressed in asthma are proteases associated with mast cells and basophils (tryptase beta 2, tryptase alpha/beta 1, and carboxypeptidase A3). This gene signature for mast cells/basophils emerges in an unbiased genome-wide expression profile of the asthmatic epithelial compartment. Thus, mast cells and basophils, which secrete proteases, cytokines and lipid mediators, and are well equipped to induce epithelial cell activation,^{13, 14} are likely to have important roles in the pathophysiology of the specific epithelial dysfunction that characterizes asthma.

We identified 30 genes that were up- or down-regulated at least two-fold with inhaled corticosteroids. CST1 was all included in this group of genes. Notably, high expression of these genes in the airway epithelium at baseline predicted corticosteroid-induced improvements in airflow at 4 weeks, implying that activation of these pathways is characteristic of a steroid-responsive asthma phenotype. Although airway epithelial expression of these genes is unlikely to form the basis of a diagnostic test (given barriers to sampling these cells in clinical practice), the pathological processes mediated by these genes may be useful in defining phenotypes of asthma in research studies and may identify more selective drug targets in the treatment of asthma.

PRR4 (entrez gene ID # 11272, listed in Table 1): This gene is induced in asthma in our microarray data (3.9 fold). It is not sensitive to steroids (no significant change in the fluticasone arm as compared to the placebo arm of the study).

HDAC9 (entrez gene ID # 9734): This gene was induced in asthma by taqman (60% increased) and sensitive to steroids by taqman (2-fold reduction).

CST1 (entrez gene ID # 1469): This gene was induced in asthma by taqman (50-fold) and in our microarray array (4-fold). It was also sensitive to steroids (2-fold reduction by array and 6-fold reduction by taqman in the fluticasone arm as compared to the placebo arm of the study).

Definitions

Unless otherwise stated, the following terms used in the specification and claims have the meanings given below.

It is noted here that as used in this specification and the appended claims, the singular forms “a,” “an,” and “the” include plural reference unless the context clearly dictates otherwise.

“Asthma mediator” refers to a molecule that is a direct mediator of asthma, as well as a mediator of steroid responsiveness and/or inflammation, e.g., an indirect mediator of asthma

“Inflammatory and fibrotic diseases of the lung” refers to interstitial lung disease, fibrotic lung disease, including pulmonary fibrosis. These diseases are caused for example, by familial/genetic factors, environmental factors, infection, medication, connective tissue disease, and sarcoidosis.

“Anti-rejection drug” refers to a drug used as an immunosuppressant for organ and tissue transplantation as well as for severe skin disorders as psoriasis and such other diseases as rheumatoid arthritis, Crohn's disease (chronic inflammation of the digestive tract) and patchy hair loss (alopecia areata). Such drugs can be classified into three broad categories: (1) Cyclosporins (e.g., Neoral, Sandimmune, SangCya). These drugs act by inhibiting T-cell activation, thus preventing T-cells from attacking the transplanted organ. (2) Azathioprines (e.g., Imuran). These drugs disrupt the synthesis of DNA and RNA and cell division. (3) Corticosteroids such as prednisolone (Deltasone, Orasone). These drugs suppress the inflammation associated with transplant rejection. In one embodiment, the antirejection drug is Sirolimus (Rapamune, rapamycin). Other anti-rejection drugs include Mycopehnolate (CellCept), Glatiramer acetate (Copaxone), and tacrolimus (Prograf).

The agents for use according to the invention are agents which alter the expression, levels or activity of CST1, HDAC9 or PRR4. The agents include “modulators” and “inhibitors” of CST1, HDAC9 or PRR4. On one hand, preferred agents are inhibitors of CST1, HDAC9 or PRR4. In some embodiments, the agents are polynucleotides, polypeptides, small organic molecules, siRNA, and naturally occurring ligands and modulators of CST1, HDAC9 or PRR4, or ribozymes.

Modulators are agents which can increase or decrease a referenced activity, function or entity. Modulators include inhibitors and activators which have effects opposite to inhibitors (e.g., increase, stimulate, augment, enhance, accelerate) a referenced activity or entity. Preferred modulators for use according to the invention are inhibitors of CST1, HDAC9 or PRR4 levels or activity.

An inhibitor of CST1, HDAC9 or PRR4 levels or activity is an agent which reduces the activity or levels of CST1, HDAC9 or PRR4 in vitro or in vivo. Such modulators include, but are not limited to, respectively, an anti-CST1, HDAC9 or PRR4 antibody; an anti-CST1, HDAC9 or PRR4 siRNA molecule; an anti-CST1, HDAC9 or PRR4-ribozyme; a compound which binds to CST1, HDAC9 or PRR4 to reduce a biological activity thereof, or an agent or compound which inhibits the expression, transcription, or translation of CST1, HDAC9 or PRR4 nucleic acids in a host cell. In some embodiments, the modulators are provided in a composition also comprising a sterile carrier and/or physiologically acceptable carrier. Accordingly, a modulator or inhibitor can be small organic molecules, antibodies, peptides, lipids, peptides, cyclic peptides, nucleic acids, antisense molecules, and ribozymes.

In some embodiments, the inhibitor is a CST1, HDAC9 or PRR4 polypeptide or fragment which lacks the biological efficacy of CST1, HDAC9 or PRR4, or has a substantially reduced biological efficacy in comparison to the native proteins, and is capable of competing with the native or endogenous CST1, HDAC9 or PRR4 protein for binding to its intracellular receptors or ligands and which thereby blocks the activity of endogenous CST1, HDAC9 or PRR4.

With regard to amino acid sequence, CST1, HDAC9 or PRR4 includes the accession numbers referenced herein or one which is substantially identical thereto and/or formed by subsequent post-translational modifications as may occur naturally in a mammalian or human cell including, but not limited to, for instance, removal of the N-terminal methionine residue or glycosylation. In some embodiments, the gene is a naturally occurring variant (e.g., splice variant) or differs in one, two, or three amino acids (additions, deletions, or substitutions) therefrom. In these embodiments, the gene or protein has a functional activity. In some embodiments, the gene may be a conservatively modified variant. In some other embodiments still, the polypeptide sequence can be that of a mammal including, but not limited to, primate, e.g., human; rodent, e.g., rat, mouse, hamster; cow, pig, horse, sheep. The proteins of the invention include both naturally occurring or recombinant molecules. In some embodiments, the amino acids of the polypeptide are all naturally occurring amino acids as set forth below. In other embodiments, one or more amino acids may be substituted by an artificial chemical mimetic of a corresponding naturally occurring amino acids.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Methods for obtaining (e.g., producing. isolating, purifying, synthesizing, and recombinantly manufacturing) polypeptides are well known to one of ordinary skill in the art.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to “conservatively modified variants” of amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

An antibody according to the invention is an antibody which can specifically bind to CST1, HDAC9 or PRR4. The antibodies for use according to the invention include, but are not limited to, recombinant antibodies, polyclonal antibodies, monoclonal antibodies, chimeric antibodies, human monoclonal antibodies, humanized or primatized monoclonal antibodies, and antibody fragments.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}—C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An “siRNA” or “RNAi” refers to a nucleic acid that forms a double stranded RNA, which double stranded RNA has the ability to reduce or inhibit expression of a gene or target gene when the siRNA expressed in the same cell as the gene or target gene. “siRNA” or “RNAi” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length.

The design and making of siRNA molecules and vectors are well known to those of ordinary skill in the art. For instance, an efficient process for designing a suitable siRNA is to start at the AUG start codon of the mRNA transcript (e.g., see, FIG. 5) and scan for AA dinucleotide sequences (see, Elbashir et al. EMBO J. 20: 6877-6888 (2001). Each AA and the 3′ adjacent nucleotides are potential siRNA target sites. The length of the adjacent site sequence will determine the length of the siRNA. For instance, 19 adjacent sites would give a 21 Nucleotide long siRNA siRNAs with 3′ overhanging UU dinucleotides are often the most effective. This approach is also compatible with using RNA pol III to transcribe hairpin siRNAs. RNA pol III terminates transcription at 4-6 nucleotide poly(T) tracts to create RNA molecules having a short poly(U) tail. However, siRNAs with other 3′ terminal dinucleotide overhangs can also effectively induce RNAi and the sequence may be empirically selected. For selectivity, target sequences with more than 16-17 contiguous base pairs of homology to other coding sequences can be avoided by conducting a BLAST search (see, www.ncbi.nlm.nih.gov/BLAST).

The siRNA can be administered directly or an siRNA expression vectors can be used to induce RNAi. A vector can have inserted two inverted repeats separated by a short spacer sequence and ending with a string of T's which serve to terminate transcription. The expressed RNA transcript is predicted to fold into a short hairpin siRNA. The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary. A preferred order of the siRNA expression cassette is sense strand, short spacer, and antisense strand. Hairpin siRNAs with these various stem lengths (e.g., 15 to 30) are suitable. The length of the loops linking sense and antisense strands of the hairpin siRNA can have varying lengths (e.g., 3 to 9 nucleotides, or longer). The vectors may contain promoters and expression enhancers or other regulatory elements which are operably linked to the nucleotide sequence encoding the siRNA.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers. These control elements may be designed to allow the clinician to turn off or on the expression of the gene by adding or controlling external factors to which the regulatory elements are responsive.

In some embodiments, the CST1, HDAC9 or PRR4 inhibitor is CST1, HDAC9 or PRR4 ribozyme which can inhibit the expression of CST1, HDAC9 or PRR4, respectively, when present in a cell. Ribozymes are enzymatic RNA molecules capable of catalysing the specific cleavage of RNA. The mechanism of ribozyme action involves sequence specific hybridisation of the ribozyme molecule to complementary target RNA, followed by an endonucleolytic cleavage. Within the scope of the invention are engineered hammerhead motif ribozyme molecules that specifically and efficiently catalyse endonucleolytic cleavage of CST1, HDAC9 or PRR4 mRNA. Specific ribozyme cleavage sites within any potential RNA target are initially identified by scanning the target molecule for ribozyme cleavage sites, which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. Both anti-sense RNA and DNA molecules and ribozymes of the invention may be prepared by any method known in the art for the synthesis of RNA molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors, which incorporate suitable RNA polymerase promoters such as the T7 or SP6 polymerase promoters. Methods of making ribozymes are well known in the art (see, for instance, U.S. Patent Application Publication No. 20060062785).

Construction of suitable vectors for the siRNA or Ribozymes containing the desired siRNA or Ribozyme sequences and control sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and religated in the form desired.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

The terms “identical” or percent “identity,” in the context of two or more nucleic acids or polypeptide sequences, including siRNA and polypeptides, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to the full length of the reference sequence, usually about 25 to 100, or 50 to about 150, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

The antibody or polypeptides for use according to the invention can have a label or detectable moiety attached thereto. A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “agent” “test compound” or “candidate molecule” or “modulator” or grammatical equivalents as used herein describes any molecule, either naturally occurring or synthetic, e.g., protein, polypeptide, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, RNAi, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

“Determining the functional effect” refers to assaying for a compound that increases or decreases a parameter that is indirectly or directly under the influence of a polynucleotide or polypeptide for use according to the invention, e.g., measuring physical and chemical or phenotypic effects. Such functional effects can be measured by any means known to those skilled in the art, e.g., changes in spectroscopic (e.g., fluorescence, absorbance, refractive index), hydrodynamic (e.g., shape), chromatographic, or solubility properties for the protein; measuring inducible markers or transcriptional activation of the protein; measuring binding activity or binding assays, e.g. binding to antibodies; measuring changes in ligand binding affinity; e.g., via chemiluminescence, fluorescence, colorimetric reactions, antibody binding, inducible markers, and ligand binding assays.

Samples or assays for identifying the modulators according to the invention are conducted in the presence of the candidate inhibitor and then the results are compared to control samples without the modulator to examine for the desired activity or to determine the functional effect of the candidate inhibitor. A positive reference control which is an agent having the desired activity may be used. In the case of CST1, HDAC9 or PRR4 polypeptides, the positive control agent may be the native peptides themselves. Control samples (untreated with inhibitors) are assigned a relative of 100%. Inhibition is achieved when the activity value relative to the control is about 80%, preferably 50%, more preferably 25 to 0%. Suitable methods for identifying inhibitors for use according to the invention are well known in the art and are further exemplified in the Examples.

The term “isolated” with regard to polypeptide or peptide fragment or nucleotides as used herein refers to a polypeptide or a peptide fragment or polynucleotide which either has no naturally-occurring counterpart or has been separated or purified from components which naturally accompany it, e.g., in normal tissues such as lung, kidney, or placenta, tumor tissue such as colon cancer tissue, or body fluids such as blood, serum, or urine. Typically, the polypeptide or peptide fragment or polynucleotide is considered “isolated” when it is at least 70%, by dry weight, free from the proteins and other naturally-occurring organic molecules with which it is naturally associated. Preferably, a preparation of a polypeptide (or peptide fragment thereof) or polynucleotide of the invention is at least 80%, more preferably at least 90% or 95%, and most preferably at least 99%, by dry weight, the polypeptide (or the peptide fragment thereof), or polynucleotide, respectively, of the invention. Thus, for example, a preparation of polypeptide x is at least 80%, more preferably at least 90%, and most preferably at least 99%, by dry weight, polypeptide x. Since a polypeptide or polynucleotide that is chemically synthesized is, by its nature, separated from the components that naturally accompany it, the synthetic polypeptide is “isolated.”

An isolated polypeptide (or peptide fragment) or polynucleotide of the invention can be obtained, for example, by extraction from a natural source (e.g., from tissues or bodily fluids); by expression of a recombinant nucleic acid encoding the polypeptide; or by chemical synthesis. A polypeptide or polynucleotide that is produced in a cellular system different from the source from which it naturally originates is “isolated,” because it will necessarily be free of components which naturally accompany it. The degree of isolation or purity can be measured by any appropriate method, e.g., column chromatography, polyacrylamide gel electrophoresis, or HPLC analysis.

Assays for Modulators of CST1, HDAC9 OR PRR4

Assays

Modulation of CST1, HDAC9 or PRR4 levels and activity, and corresponding modulation of epithelial cell activation and/or migration, can be assessed using a variety of in vitro and in vivo assays, including cell-based models. The assays can be used to test for inhibitors and activators of CST1, HDAC9 or PRR4 or fragments thereof, and, consequently, inhibitors and activators of epithelial cell. Modulators can be tested using either recombinant or naturally occurring proteins, preferably human.

Epithelial cell migration can be measured using time-lapse migration microscopy. The cells can be plated on Delta-T glass dishes (0.5 mm; Bioptechs Inc., Butler, Pa.) that have been coated with PN. One and a half hours after plating the cells, the medium can be refreshed and cell migration monitored from images captured at 20-min interval from a Nikon Diaphot microscope equipped with a digital camera. The positions of the nuclei can be tracked to measure cell movement. Cell velocity can be calculated in micrometers per 8 h using the Image-Pro software (Media Cybernetics, Silver Springs, Md.). See, Gillan et al., Cancer Research 62, 5358-5364 (2002).

Measurement of activation, migration, inhibition or loss-of-migration phenotype can be performed using a variety of assays, in vitro, in vivo, and ex vivo. A suitable physical, chemical or phenotypic change that affects activity or binding can be used to assess the influence of a test compound on the polypeptide of this invention. When the functional effects are determined using intact cells or animals, one can also measure a variety of effects such as effects or respiratory tract function, hormone release, transcriptional changes to both known and uncharacterized genetic markers (e.g., northern blots), cellular movement towards a ligand, movement of labeled cells, changes in cell metabolism such as pH changes, and changes in intracellular second messengers such as Ca2+, IP3, cGMP, or cAMP; as well as changes related to activation and migration, e.g., cellular proliferation, cell surface marker expression, and apoptosis as known to one of ordinary skill in the art.

In Vitro Assays

Assays to identify compounds with modulating activity can be performed in vitro. Such assays can used the full length proteins or a variant or fragment thereof. As described below, the binding assay can be either solid state or soluble. Preferably, the protein, fragment thereof or membrane is bound to a solid support, either covalently or non-covalently. Often, the in vitro assays of the invention are ligand binding or ligand affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein.

In one embodiment, a high throughput binding assay is performed in which the protein or fragment thereof is contacted with a potential modulator and incubated for a suitable amount of time. In one embodiment, the potential modulator is bound to a solid support, and the protein is added. In another embodiment, the protein is bound to a solid support. A wide variety of modulators can be used, as described below, including small organic molecules, peptides, antibodies, and ligand analogs. A wide variety of assays can be used to identify-modulator binding, including labeled protein-protein binding assays, electrophoretic mobility shifts, immunoassays, enzymatic assays such as phosphorylation assays, and the like. In some cases, the binding of the candidate modulator is determined through the use of competitive binding assays, where interference with binding of a known ligand is measured in the presence of a potential modulator. Ligands for the proteins are known. Either the modulator or the known ligand is bound first, and then the competitor is added. After the protein is washed, interference with binding, either of the potential modulator or of the known ligand, is determined. Often, either the potential modulator or the known ligand is labeled.

Cell-Based in Vivo Assays

In another embodiment, the CST1, HDAC9 or PRR4 protein is expressed in a cell, and functional, e.g., physical and chemical or phenotypic, changes are assayed to identify the modulatory effects. Cells expressing the proteins can also be used in binding assays. Any suitable functional effect can be measured, as described herein. For example, ligand binding, cell surface marker expression, cellular proliferation, apoptosis, cytokine production, transduction, e.g., changes in intracellular Ca2+ levels, are all suitable assays to identify potential modulators using a cell based system. Suitable cells for such cell based assays include both primary lymphocytes and epithelial cell lines as described herein. The protein can be naturally occurring or recombinant. Also, as described above, fragments of the proteins or chimeras of same can be used in cell based assays.

In another embodiment, cellular proliferation, migration, or apoptosis can be measured using 3H-thymidine incorporation or dye inclusion. Cytokine production can be measured using an immunoassay such as an ELISA.

In another embodiment, cellular polypeptide levels are determined by measuring the level of protein or mRNA. The level of protein or are measured using immunoassays such as western blotting, ELISA and the like with an antibody that selectively binds to the polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, expression can be measured using a reporter gene system. Such a system can be devised using a CST1, HDAC9 or PRR4 protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

Animal Models

Animal models of lymphocyte activation and migration also find use in screening for modulators of lymphocyte activation or migration. Similarly, transgenic animal technology including gene knockout technology, for example as a result of homologous recombination with an appropriate gene targeting vector, or gene overexpression, will result in the absence or increased expression of the CST1, HDAC9 or PRR4 protein. The same technology can also be applied to make knock-out cells. When desired, tissue-specific expression or knockout of the protein may be necessary. Transgenic animals generated by such methods find use as animal models of lymphocyte activation and migration and are additionally useful in screening for modulators of lymphocyte or epithelial cell activation and migration.

Knock-out cells and transgenic mice can be made by insertion of a marker gene or other heterologous gene into an endogenous gene site in the mouse genome via homologous recombination. Such mice can also be made by substituting an endogenous with a mutated version of the gene, or by mutating an endogenous, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

Modulators

The compounds tested as modulators of the CST1, HDAC9 or PRR4 can be any small organic molecule, or a biological entity, such as a protein, e.g., an antibody or peptide, a sugar, a nucleic acid, e.g., an antisense oligonucleotide or a ribozyme, or a lipid. Alternatively, modulators can be genetically altered versions of the CST1, HDAC9 or PRR4 protein. Typically, test compounds will be small organic molecules, peptides, lipids, and lipid analogs.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, R U, 3D Pharmaceuticals, Exton, Pa., Martek Biosciences, Columbia, Md., etc.).

Solid State and Soluble High Throughput Assays

In one embodiment the invention provides soluble assays using a CST1, HDAC9 or PRR4 protein, or a cell or tissue expressing the protein, either naturally occurring or recombinant. In another embodiment, the invention provides solid phase based in vitro assays in a high throughput format, where the protein or fragment thereof is attached to a solid phase substrate. Any one of the assays described herein can be adapted for high throughput screening, e.g., ligand binding, cellular proliferation, cell surface marker flux, e.g., CD-69, screening, radiolabeled GTP binding, second messenger flux, e.g., Ca2+, IP3, cGMP, or cAMP, cytokine production, etc.

In the high throughput assays of the invention, either soluble or solid state, it is possible to screen up to several thousand different modulators or ligands in a single day. This methodology can be used for proteins in vitro, or for cell-based or membrane-based assays comprising an protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

Methods of Treatment

The terms “treating” or “treatment” include:

(1) preventing the disease, i.e., causing the clinical symptoms of the disease not to develop in a mammal that may be exposed to the organism but does not yet experience or display symptoms of the disease,

(2) inhibiting the disease, i.e., arresting or reducing the development of the disease or its clinical symptoms,

(3) relieving the disease, i.e., causing regression of the disease or its clinical symptoms.

In some embodiments, the treatments, on an acute or chronic basis, reduce the frequency, severity, or recurrence of asthmatic attacks, reduce shortness of breath, cough, or wheezing, improve tissue oxygenation, decrease resistance to air flow in the respiratory tract, increase vital capacity, forced expiratory air volume, peak expiratory air flows, reduce collagen deposition in the respiratory tract, reduce vascular remodeling in the respiratory tract, or smooth muscle proliferation in the respiratory tract in subjects having asthma.

In one aspect, the invention provides a method of treating asthma, allergic rhinitis, chronic obstructive pulmonary disease, or inflammatory or fibrotic diseases of the lung in a subject, or asthma, allergic rhinitis, chronic obstructive pulmonary disease, or inflammatory or fibrotic diseases of the lung in a subject or enhancing the effects of corticosteroids in treating these conditions, the method comprising the step of administering to a subject an anti-rejection drug that modulates CST1, HDAC9 or PRR4. Preferably the drug is formulated for administration by inhalation.

Diagnosis of Asthma

Methods of diagnosing asthma, allergic rhinitis, and other inflammatory or fibrotic disease of the lung, and obstructive diseases are well known to persons of ordinary skill in the art. For example, spirometry can be used to assess lung function. The diagnosis of asthma, in particular, may be made in part based upon family history or personal history of a severe and sudden episode or recurrent episodes of wheezing, coughing or shortness of breath which may be associated with exposure to an allergen or exacerbated or precipitated by moderate exercise. Typically a physical exam is involved to detect the disease.

Using a nasal speculum, the nose may be examined for signs of allergic disease such as increased nasal secretions, swelling or polyps which may be triggering asthma. A stethoscope may be used to listen to the sounds the lungs make during breathing. Wheezing sounds are one of the main indicators of the obstructed airways associated with asthma. In addition, allergic conditions such as eczema or hives, which are often associated with asthma.

Pulmonary function tests are particularly useful in confirming the diagnosis of respiratory diseases. These tests include spirometry to determine vital capacity, the maximum amount of air that you can inhale and exhale; the peak expiratory flow rate, also known as the peak flow rate, which is the maximum flow rate you can generate during a forced exhalation; and forced expiratory volume, which is the maximum amount of air you can exhale in one second.

If the measurements are below normal for a person your age, a bronchodilator drug used in asthma treatment can be administered to open obstructed air passages and the spirometry repeated. If the measurements improve significantly, asthma is likely.

In addition, asthma may be diagnosed by challenging the individual with exercise, or by inhaling an airway-constricting chemical or taking several breaths of cold air. After the challenge with a symptom-producing substance or activity, the spirometry test is readministered. If the spirometry measurements fall significantly, asthma is indicated.

In certain aspects, the present invention provides methods of diagnosing or providing a prognosis for asthma, allergic rhinitis, COPD, and inflammatory and fibrotic disease of the lung. As used herein, the term “providing a prognosis” refers to providing a prediction of the probable course and outcome of a cancer or the likelihood of recovery from the disease.

Diagnosis or prognosis can involve determining the level of CST1, HDAC9 or PRR4 expression (i.e., transcription or translation) in a patient and then comparing the level or localization to a baseline or range. Typically, the baseline value is representative of CST1, HDAC9 or PRR4 expression levels in a healthy person. Variation of levels of a polypeptide or polynucleotide of the present invention from the baseline range (i.e., either up or down) indicates that the patient has a lung disease described herein or is at risk at developing it. In some embodiments, the level of CST1, HDAC9 or PRR4 expression is measured by taking a blood or tissue sample from a patient and measuring the amount of a polypeptide or polynucleotide of the present invention in the sample using any number of detection methods, such as those discussed herein.

Any antibody-based technique for determining a level of expression of a protein of interest can be used to measure the level of CST1, HDAC9 or PRR4 expression in the sample. For example, immunoassays such as ELISA assays, immunoprecipitation assays, and immunohistochemical assays can be used to detect differential protein expression in patient samples. One skilled in the art will know of additional antibody-based techniques that can be used for determining a level of CST1, HDAC9 or PRR4 expression according to the methods of the present invention. PCR assays can be used to detect expression levels of nucleic acids, as well as to discriminate between variants in genomic structure, such as insertion/deletion mutations, truncations, or splice variants.

In some embodiments, the expression of CST1, HDAC9 or PRR4 in tissue may be evaluated by visualizing the presence and/or localization of CST1, HDAC9 or PRR4 in the subject. Any technique known in the art for visualizing tissues or organs in live subjects can be used in the methods of the present invention.

Pharmaceutical Compositions.

When used for pharmaceutical purposes, the agents used according to the invention are typically formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966). The compositions can additionally include a stabilizer, enhancer, or other pharmaceutically acceptable carriers or vehicles. A pharmaceutically acceptable carrier can contain a physiologically acceptable compound that acts, for example, to stabilize the nucleic acids or polypeptides of the invention and any associated vector. A physiologically acceptable compound can include, for example, carbohydrates, such as glucose, sucrose or dextrans; antioxidants, such as ascorbic acid or glutathione; chelating agents; low molecular weight proteins or other stabilizers or excipients. Other physiologically acceptable compounds include wetting agents, emulsifying agents, dispersing agents, or preservatives, which are particularly useful for preventing the growth or action of microorganisms. Various preservatives are well known and include, for example, phenol and ascorbic acid. Examples of carriers, stabilizers, or adjuvants can be found in Remington's Pharmaceutical Sciences, Mack Publishing Company, Philadelphia, Pa., 17th ed. (1985).

The pharmaceutical compositions according to the invention comprise a therapeutically effective amount of an agent (e.g., polypeptide, antibody, si RNA, or ribozyme) according to the invention and a pharmaceutically acceptable carrier. By “therapeutically effective dose or amount” herein is meant a dose that produces effects for which it is administered (e.g., treatment or prevention of asthma). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). The agent, if a salt, is formulated as a “pharmaceutically acceptable salt.”

A “pharmaceutically acceptable salt” or to include salts of the active compounds which are prepared with relatively nontoxic acids or bases, according to the route of administration. When inhibitors of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Suitable formulations for use in the present invention are found in Remington: The Science and Practice of Pharmacy, 20th Edition, Gennaro, Editor (2003) which is incorporated herein by reference. Moreover, for a brief review of methods for drug delivery, see, Langer, Science (1990) 249:1527-1533, which is incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

For injection, the compounds of the present invention can be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the inhibitors for use according to the invention can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

The compositions containing the agents of the invention (e.g., antibodies, small organic molecules, polypeptides, siRNA, ribozymes) can be administered for therapeutic or prophylactic treatments. In therapeutic applications, compositions are administered to a patient having asthma in a “therapeutically effective dose.” Single or multiple administrations of the compositions may be administered depending on the dosage and frequency as required and tolerated by the patient. A “patient” or “subject” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

The pharmaceutical compositions can comprise additional active agents, including additional agents useful in treating asthma.

As used herein, the term “carrier” refers to a typically inert substance used as a diluent or vehicle for an active agent to be applied to a biological system in vivo or in vitro. (e.g., drug such as a therapeutic agent). The term also encompasses a typically inert substance that imparts cohesive qualities to the composition.

The agents may also formulated in unit dosage form effective for the treatment of asthma. The dosage of the agent depends upon many factors that are well known to those skilled in the art, for example, the particular compound; the condition being treated; the age, weight, and clinical condition of the recipient patient; and the experience and judgment of the clinician or practitioner administering the therapy. An effective amount of the compound is that which provides either subjective relief of symptoms or an objectively identifiable improvement as noted by the clinician or other qualified observer. The dosing range varies with the compound used, the route of administration and the potency of the particular compound.

In yet other embodiments, the invention provides topical sustained and prolonged release pharmaceutical compositions comprising one or more pharmacological compounds described supra, and a pharmaceutically acceptable carrier, to treat asthma. Preferably, the compositions are administered in unit dosage form to a subject in need of such treatment.

Pulmonary Administration

Inhalable Powders

In some embodiments, the agents are administered directly to the lung by inhalation. Accordingly, the agents for use according to the invention may be formulated as inhalable powders in admixture with suitable physiologically acceptable excipients (see, U.S. Patent No. 20060034776 which is incorporated herein by reference with respect to suitable methods of administering pharmaceutical agents by inhalation).

Propellant Gas-Driven Inhalation Aerosols

Inhalation aerosols containing propellant gas according to the invention may contain the agents for use according to the invention dissolved in the propellant gas or in dispersed form. The propellant gases which may be used to prepare the inhalation aerosols according to the invention are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG134a, TG227, and mixtures thereof. The propellant-driven inhalation aerosols according to the invention may also contain other ingredients such as cosolvents, stabilizers, surfactants, antioxidants, lubricants, preservatives and pH adjusters. All these ingredients are known in the art. When in dispersed form, the agents can, for instance, be formulated to have an average particle size of up to 10 microns or preferably from 0.1 to 5 microns, or from 1 to 5 microns.

Propellant-Free Inhalable Solutions or Suspensions

Propellant-free inhalable solutions and suspensions of the agents for use according to the invention are contemplated. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water.

### EXAMPLES

The following examples are offered to illustrate, but not to limit the claimed invention.

### Example 1

### Contribution of Aberrant Gene Expression by Airway Epithelial Cells to Asthma Pathophysiology

Microarrays, which now provide genome-wide coverage, were incorporated microarrays in a clinical study to identify novel epithelial mechanisms in asthma and markers of corticosteroid responsiveness.

Methods

Human Subjects and Protocols

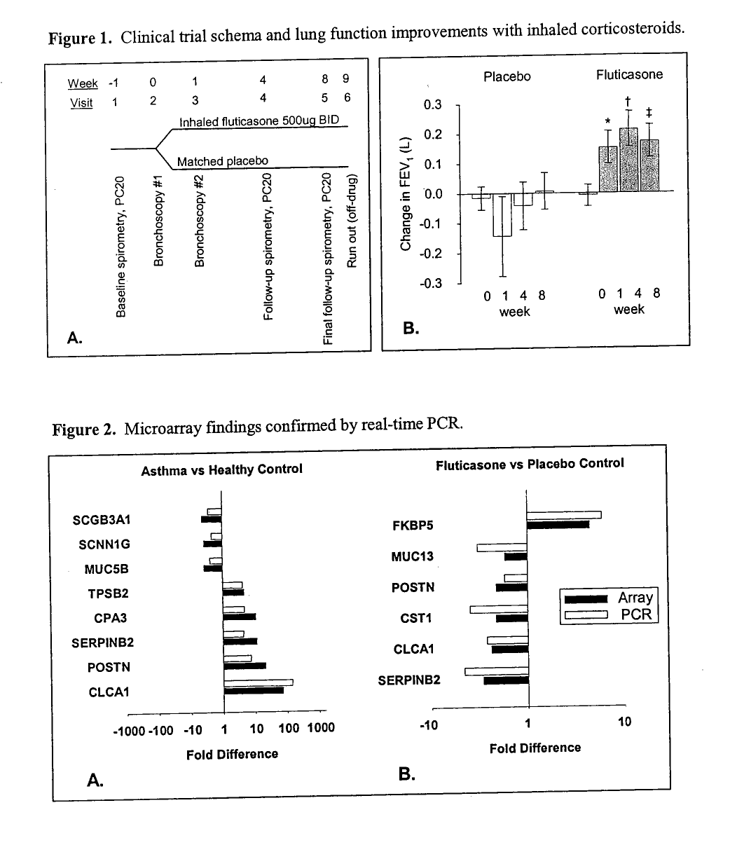

In three cross sectional studies and a clinical trial, epithelial brushings were collected by bronchoscopy from 42 subjects with asthma, 16 current smokers without asthma, and 28 healthy controls. Cross-sectional studies comprised 2 visits, the first for characterization and the second for bronchoscopy. The clinical trial was a 10 week, randomized, double-blind, placebo-controlled trial specifically designed to determine the effects of inhaled fluticasone (500 μg twice daily) on airway gene expression and to relate gene expression changes to improvements in lung function (FIG. 1). Bronchoscopy was performed at baseline and repeated 1 week after starting study drug. Thirty-five subjects had adequate baseline bronchoscopy and 32 had RNA available from epithelial brushings at both bronchoscopies. Lung function was measured at intervals over the 8-week treatment period. Epithelial RNA from a subset of the subjects studied by microarray here have been used in other PCR based gene expression studies performed by our group.⁴ Additional information is provided in the supplementary appendix.

Gene Expression Analysis

Brushings yielded 1,300,000±800,000 cells of which 97±3% were epithelial. For microarray analysis, 25 μg total epithelial RNA was amplified using NuGen Ovation RNA amplification system. Then 2.75 μg sscDNA was hybridized to Affymetrix U133 plus 2.0 arrays (>54,000 probe sets coding for 38,500 genes). Array images were analyzed using Affymetrix GeneChip Expression Analysis Software. Bioconductor⁵ was used for quality control (affyPLM algorithm), preprocessing (RMA algorithm), cluster analysis, and linear modeling.⁵⁻⁷ Raw array data are available from the Gene Expression Omnibus public database (http://www.ncbi.nlm.nih.gov/geo/, accession number pending). Selected microarray findings were confirmed by real-time PCR, enzyme linked immunosorbent assay (ELISA) or immunohistochemistry. In vitro studies of the effects of interleukin-13 (IL-13) on expression of selected genes were performed using primary airway epithelial cells grown at an air-liquid interface (additional detail in supplementary appendix).

Statistical Analysis

For between group comparisons, t-test, rank-sum test or ANOVA followed by pair-wise analyses with Sidak correction were used as appropriate. Correlation of mRNA copy number by PCR (log transformed for normality) and lung function was performed using Pearson correlation. STATA version 9 software was used (StataCorp, College Station Tex.), and p<0.05 was taken as statistically significant. In microarray analyses, differential gene expression was assessed using linear models (controlling for age and gender). For between group comparisons, a Bonferroni corrected p-value<0.05 was used to identify differentially expressed genes. For the placebo-controlled trial, changes in gene expression from baseline to post-intervention were calculated and the mean changes in the fluticasone and placebo groups were compared. A two-fold change (either higher or lower in the fluticasone group as compared to placebo), was used to identify differentially expressed genes. Microarray analyses were performed using Bioconductor open source software⁵ in the R statistical environment.

Results

Subjects

The age and gender of asthmatic and healthy subjects were similar. The smokers were all currently smoking (43±28 pack per day), were older than the healthy and asthmatic subjects and more likely to be male (Table 1). Lung function was moderately reduced in both smokers and asthmatics.

Clinical Trial

Of 32 subjects with evaluable brushings from both bronchoscopies (see supplementary appendix), 19 were randomized to fluticasone and 13 to placebo (FIG. 1a). Subjects randomized to fluticasone had greater improvements in FEV₁ than placebo by one week which were sustained at 4 weeks and 8 weeks (FIG. 1b). Although subjects randomized to fluticasone demonstrated within group improvements in PC₂₀ methacholine at 4 and 8 weeks (p<0.003), the PC₂₀ improvements were not statistically different for fluticasone versus placebo groups (FIG. 4, supplementary appendix).

Epithelial Gene Expression

Asthma vs Healthy:

Using the conservative Bonferroni correction for multiple comparisons, differential expression was discovered for 26 probe sets representing 22 genes (Table 1s, supplementary appendix) (13 genes were increased and 9 were decreased). The gene most highly induced was CLCA1 (6.2-fold induced), a putative chloride channel which is known to be upregulated in asthma based on the literature⁸⁻¹¹ and on PCR analyses performed by our group using some of the samples which were studied by microarray here⁴ (see supplementary appendix). Other upregulated genes in asthma included periostin (4.4-fold-induced), plasminogen activator inhibitor-2 (serpinB2, 3.5-fold-induced), and mast cell markers such as carboxypeptidase A3 (3.4-fold-induced), tryptase beta 2 (2.2-fold induced) and tryptase alpha/beta 1 (2.1-fold-induced). Eight genes were further evaluated by quantitative real-time PCR and their differential expression in asthma was confirmed (FIG. 2a).

Smokers vs Healthy:

We found differential expression for 54 probe sets representing 40 genes (Table 6, supplementary appendix) (27 genes increased and 13 decreased). Twelve of the 27 upregulated genes display oxidoreductase activity, including AKR1B10 (13.6-fold induced), CYP1B1 (10.4-fold induced), ALDH3A1 (5.1-fold induced), GPX2 (4.9-fold induced), AKR1C2 (3.9-fold induced) and ME1 (3.4-fold induced).

Transmembrane protein 45 (TMEM45A), a gene of uncertain function, was the only gene differentially expressed in both asthma vs healthy and smokers vs healthy comparisons (repressed by 2.0-fold in asthma and 2.8-fold in smokers).

Corticosteroid Responsive Genes

Overall, 33 probe sets representing 30 genes demonstrated a 2-fold change in expression in the fluticasone group as compared to the placebo group (Table 4s, supplementary appendix). The most highly induced gene with a known identity on the microarray was FK506 binding protein 51 (FKBP51) (FIG. 2b). The expression levels of 6 genes were further evaluated by quantitative real-time PCR. Differential expression by PCR was similar to that measured by microarray (FIG. 2b) with statistically significant changes for all genes except periostin (POSTN) which showed a strong trend (p=0.06) for reduced expression after corticosteroids.

In order to identify genes that are both associated with asthma AND corticosteroid-responsive, we compared the 22 genes differentially expressed in asthma (Table 1 s) with the 30 corticosteroid sensitive genes (Table 4s). Three genes met both criteria —CLCA1, periostin, and serpinB2. All three genes were highly up-regulated in asthma and down-regulated by corticosteroids.

Gene Expression and Lung Function

Since transcript levels for CLCA1, periostin, and serpinB2 were markedly increased in asthmatic subjects and responsive to corticosteroids, we explored the possibility that baseline expression levels or changes in the expression of these genes (log copy number by real time RT-PCR) might be associated with improvements in lung function in the fluticasone group. We found that baseline expression of CLCA1, periostin, and serpinB2 correlated with improvements in FEV₁ in the corticosteroid treated subjects at 4 weeks (Table 2) and that decreases in serpinB2 expression (difference between log mRNA copy number at the second versus the first bronchoscopy) correlated with improvements in FEV₁ at both 4 and 8 weeks (r=−0.62, p=0.01 and r=−0.65, p=0.009 respectively).

FKBP51 is known to regulate the effects of corticosteroids.¹² Therefore, we examined the relationship between FKBP51 expression (as measured by real time RT-PCR) and improvements in airflow with fluticasone. Although we did not find significant correlations between change in FKBP51 expression and changes in FEV₁, we did find that higher baseline expression of FKBP51 correlated with poor FEV₁ response to fluticasone (Table 2 and FIG. 5).

Protein Confirmation

Periostin and serpin B2 have not previously been associated with asthma, and we examined their expression at the protein level. Using ELISA (American Diagnostica, Stamford Conn.) we found that serpinB2 levels in bronchoalveaolar lavage were below the lower limit of detection (<5 ng/mL) in 13 of 13 healthy control subjects but were detectable in 13 of 27 asthmatics (median 0.05, range 0-3.3 ng/mL, p=0.002). However, in asthmatics, serpinB2 levels increased in all subjects at the second bronchoscopy (Table 4s, supplementary appendix). The increase in serpinB2 at the second bronchoscopy likely reflects a pro-coagulation response to the minor bleeding that accompanied bronchial biopsy during the first bronchosocopy.

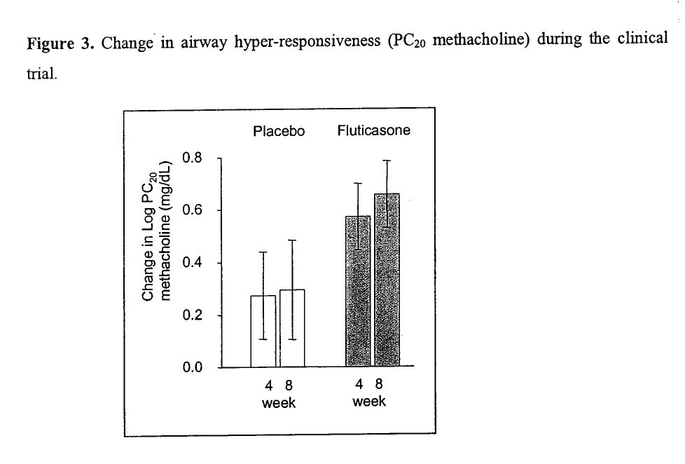

Periostin protein expression was confirmed by immunohistochemistry in bronchial biopsies from a subset of asthmatic and healthy control subjects. Periostin immunolocalized to the subepithelium in all subjects, and quantification confirmed increased expression in asthma (FIG. 3). The degree of immunostaining in asthmatics was not decreased by one week of treatment with fluticasone (FIG. 6, supplementary appendix).

IL-13 Stimulation of Airway Epithelial Cells In Vitro

To confirm that airway epithelial cells are a source of serpinB2 and periostin and that these genes may be induced by cytokines relevant to asthma, we stimulated primary human airway epithelial cells in vitro with IL-13 (0.1 to 10 ng) for up to 4 days (methods in supplementary appendix). We found that IL-13 (10 ng/mL) induced large increases in epithelial gene expression for both serpinB2 and periostin (FIG. 7, supplementary appendix).

TABLE 1
Subject Characteristics

| | Controls | Asthmatics | Smokers |
|---|---|---|---|
| n | 28 | 42 | 16 |
| Gender | 16F/12M | 25F/17M | 2F/14M* |
| Age | 36 ± 9 | 36 ± 12 | 52 ± 9* |
| FEV1 (% predicted) | 107 ± 13% | 87 ± 12%* | 83 ± 17%* |
| FEV1/FVC | 0.81 ± 0.07 | 0.72 ± 0.08* | 0.65 ± 0.13* |
| PC₂₀ (mg/dl methacholine) | 64 (64, 64) | 0.43 (0.14, 1.11) * | 19.5 (1.8, 47) * |
| GOLD classification | | | |
| Class 0 | — | — | 9 |
| Class 1 | — | — | 2 |
| Class 2 | — | — | 5 |
| DLCO (% predicted) | — | — | 87 ± 16% |
| # with DLCO <80% predicted | — | — | 6/16 |

Data are presented as mean ±SD or median (interquartile range). Abbreviations: FEV₁, forced expiratory volume in the first second; FVC, forced vital capacity; PC₂₀, the concentration of methacholine that caused a 20% decline in FEV₁; DLCO, diffusing capacity to carbon monoxide *p<0.05 compared to non-smoking healthy control subjects based on t-test (PC₂₀ log transformed for analysis). GOLD classification denotes presence and severity of COPD.²⁸ No subjects with asthma were using corticosteroids or long acting beta-agonists prior to enrollment.

TABLE 2
Correlations between gene expression and lung function in subjects
randomized to inhaled corticosteroids in the clinical trial.

| | | |
|---|---|---|
| | Improvement in FEV₁ | Improvement in FEV₁ |
| Gene | baseline to 4 weeks | baseline to 8 weeks |

Baseline mRNA expression levels*

| | | |
|---|---|---|
| CLCA1 | r = 0.60 | r = 0.47 |
| | p = 0.011 | p = 0.64 |
| Periostin | r = 0.49 | r = 0.023 |
| | p = 0.048 | p = 0.38 |
| SerpinB2 | r = 0.53 | r = 0.048 |
| | p = 0.027 | p = 0.059 |
| FKBP51 | r = −0.63 | r = −0.63 |
| | p = 0.007 | p = 0.009 |

Change with 1 week of treatment of inhaled steroids†

| | | |
|---|---|---|
| CLCA1 | r = −0.16 | r = −0.33 |
| | p = 0.56 | p = 0.24 |
| Periostin | r = −0.45 | r = −0.31 |
| | p = 0.08 | p = 0.26 |
| SerpinB2 | r = −0.62 | r = −0.65 |
| | p = 0.011 | p = 0.009 |
| FKBP51 | r = −0.10 | r = 0.39 |
| | p = 0.72 | p = 0.15 |

*copy number by real-time PCR, log transformed for normality
†defined as log copy number at second bronchoscopy minus log copy number at first bronchoscopy
Change in lung function defined as change in FEV₁ [mL] from baseline to the 4 and 8 week endpoints

Supplementary Appendix for Example 1

Overview of the Clinical Studies

We studied 42 non-smoking subjects with asthma, 16 current smokers without asthma, and 28 healthy non-smoking controls in clinical protocols which employed standardized recruitment methods, characterization procedures and a standard method for research bronchoscopy. Seven of the subjects with asthma were enrolled in a cross-sectional study of airway inflammation and remodeling in asthma which incorporated only one bronchoscopy. Thirty-five additional subjects with asthma were enrolled as part of a clinical trial specifically designed to identify corticosteroid-responsive genes. Each of these subjects had a baseline bronchoscopy and 32 of the 35 subjects also completed a second bronchoscopy after one week of treatment with either inhaled fluticasone propionate or matched placebo. All subjects with asthma had a prior physician diagnosis of asthma, had airway hyper-responsiveness (PC₂₀ on methacholine challenge testing <8 mg/mL) and were using only inhaled beta-agonist medications for therapy at baseline. Current smokers had >10 pack-year total consumption and were recruited for a cross-sectional study of airway inflammation and remodeling in smoking-related lung disease. Healthy controls were non-smokers with no history of lung disease and without airway hyper-responsiveness (PC₂₀ methacholine >16 mg/mL) studied as controls in one of three cross-sectional studies performed using standardized techniques: a) the cross-sectional asthma study described above, b) the smoking study described above, or c) a study protocol designed for ex vivo studies of airway epithelial biology.

Epithelial brushings from subsets of the healthy controls and asthmatics and smokers have been analyzed in two other studies in our laboratory. The first study applied real-time PCR to examine the expression of 11 human genes which are homologues of mouse genes that were found to be differentially regulated in a mouse model of airway inflammation (Table 5 s).¹ The goal of that study was to identify interleukin-13 (IL-13) responsive epithelial genes in mouse transgenic models, and one finding was that CLCA1 is increased in both mouse models and in human asthma. The second study applied real-time PCR to examine the expression specific mucin genes in the airway in smokers as compared to healthy control subjects. That study has recently been submitted for consideration by another journal (see abstract as “attachment A” below). The UCSF Committee on Human Research approved these studies and all subjects provided written informed consent. Additional information about inclusion and exclusion criteria, study design, and procedures are provided below.

Spirometry, methacholine challenge testing, measurement of diffusing capacity and bronchoscopy were performed as described previously.² At bronchoscopy, epithelial brushings were obtained randomly from either right or left lower lobe bronchial segments using four separate disposable cytology brushes (Mill Rose Laboratories; Mentor, Ohio). Other procedures performed during bronchoscopy included: (i) bronchoalveolar lavage (prior to brushings) which was performed in either the right middle lobe or lingula of the ipsilateral lung (100 mls lavage [50 mls×2] in each of two different subsegments) and (ii) 6-10 bronchial biopsies (after brushings) which were taken from 2nd through 4th order carinae in the contralateral lung.

### DETAILED DESCRIPTION OF THE CLINICAL STUDIES

Cross-Sectional Studies

Cross-Sectional Study of Asthma and Healthy Controls.

Protocol: This was a two visit study which enrolled 23 subjects with asthma and 17 healthy non-asthmatic controls. Of these subjects, 7 subjects with asthma and 11 healthy controls were unique (not studied in other protocols) and had bronchoscopy with epithelial brushings suitable for microarray analysis and were included in this manuscript.

For all subjects, inclusion criteria were age 18-55. Subjects with asthma all had a prior physician diagnosis of asthma and PC₂₀ to methacholine <8 mg/ml at visit 1, documenting bronchial hyperresponsiveness. Non-asthmatics all had a PC₂₀>16 mg/ml. Subjects with asthma were using only inhaled beta-agonist medications for therapy. No subjects were using inhaled corticosteroids or leukotriene antagonists. Exclusion criteria included a respiratory infection or asthma exacerbation within the previous 6 weeks, a significant smoking history (defined as more than 10 pack years lifetime or any cigarette smoking in the last year), significant medical problems other than asthma, or a history of treatment in the intensive care unit or intubation for acute asthma.

Subjects completed 2 visits 1 week apart. At visit 1, subjects underwent medical history and physical examination, spirometry, and methacholine challenge. At visit 2, bronchoscopy was performed with epithelial brushings, bronchoalveolar lavage and epithelial brushings using specific methods previously described.²⁻⁴

Cross-Sectional Study of Smokers and Healthy Controls.

This was a two visit study which enrolled 24 cigarette smokers (defined as current smoking of 10 cigarettes per day and a minimum history of 10 pack-years of exposure) and 19 non-smoking controls (defined as <10 pack-years of smoking with no smoking in the previous 10 years). Of these subjects, 15 smokers and 16 healthy controls were unique (not studied in other protocols) and had epithelial brushings suitable for microarray analysis and were included in this manuscript.

For all subjects, inclusion criteria were age 30-65 years and exclusion criteria were FEV₁/FVC<0.4, methacholine PC₂₀<1 mg/mL, history of asthma, recent upper respiratory tract infection, significant medical problems other than smoking-related lung disease, history of home oxygen use, or admission to an intensive care unit for respiratory failure.

Subjects completed 2 visits 1 week apart. At visit 1, subjects underwent medical history and physical examination, spirometry, and methacholine challenge. Smokers also received a 12-lead electrocardiogram and underwent single breath measurement of diffusing capacity for carbon monoxide (DL_{CO}, V Max Series, Sensormedic Corp.; Yorba Linda, Calif., USA). Subjects refrained from smoking for at least 4 hours prior to DL_{CO} testing. At visit 2, bronchoscopy was performed with epithelial brushings, bronchoalveolar lavage and endobronchial biopsies using specific methods previously described.²⁻⁴

Study of Healthy Controls for Ex Vivo Experiments.

Protocol: This was a two visit study which enrolled 9 subjects with asthma and 13 healthy non-asthmatics controls. Of these subjects, 2 healthy controls were unique (not studied in other protocols) and had bronchoscopy with epithelial brushings suitable for microarray analysis and were included in this manuscript.

Inclusion criteria were asthmatic and healthy non-asthmatic control subjects age 18-65 years. Subjects with asthma had PC₂₀ methacholine <8 mg/mL and FEV₁>60% predicted. Healthy control subjects had no history of asthma, PC₂₀ methacholine >8 mg/mL and FEV₁>80% predicted. Exclusion criteria included history of smoking in the past year or >10 total pack year history of smoking, lung disease other than asthma, history of medical illness known to increase the risk of bronchoscopy, upper respiratory tract infection in the past 6 weeks, hospitalization for asthma in the past 6 weeks, use of inhaled or oral corticosteroids in the past 6 weeks.

Subjects completed 2 visits 1 week apart. At visit 1, subjects underwent medical history and physical examination, spirometry, and methacholine challenge. At visit 2, bronchoscopy was performed with epithelial brushings, bronchoalveolar lavage and endobronchial biopsies using specific methods previously described.²⁻⁴

Double-Blind Randomized Controlled Trial of Inhaled Fluticasone in Subjects with Asthma

Protocol: This was a 6 visit, 10 week, randomized, double-blind, placebo-controlled trial specifically designed to determine the effects of inhaled fluticasone on airway gene expression and to relate those gene expression changes to improvements in lung function. After a one-week run-in/characterization period, subjects were randomized to receive 2 puffs BID of fluticasone propionate (250 μg/puff) or matching placebo for 8 weeks, followed by a one week run-out. Subjects were randomized in blocks designed to yield a greater number of subjects randomized to fluticasone as compared to placebo in order to maximize our ability to relate gene expression to lung function improvements within the intervention group. Bronchoscopy was performed after the 1 week run-in, and again 1 week after starting study drug with epithelial brushings, bronchoalveolar lavage and epithelial brushings using specific methods previously described.²⁻⁴ Thirty-two of the 35 subjects had RNA high quality available from epithelial brushings at both bronchoscopies. Nineteen subjects had been randomized to fluticasone and 13 to placebo. Spirometry was measured at baseline, before both bronchoscopies, at 4 and 8 weeks after starting study medication, and after the one week run-out according to American Thoracic Society criteria using a dry rolling-seal spirometer (Model VRS2000, PDS Instrumentation, S&M Instrument Co., Doylestown, Pa.). Methacholine responsiveness was measured at baseline and at 4 and 8 weeks after starting study medication using a five-breath dosimeter method (modified from Chai et al).⁵

Inclusion criteria:
1. age 18 to 70 years
2. PC20 methacholine ≦8.0 mg/mL
3. Asthma symptoms on at least 2 days per week, or beta-agonist use on at least 2 days per week, or FEV₁<85% predicted
4. Non-smoker (no smoking in past 12 months and ≦15 pack years)

Exclusion criteria:
1. Oral or inhaled steroid use in the past 4 weeks
2. FEV₁<60% predicted
3. Lung Disease Other than Asthma
4. URI4 weeks prior to visit 1
5. asthma exacerbation 6 weeks prior to visit 1
6. Increasing hyposensitization therapy for the past 3 months
7. Active cardiovascular disease, peptic ulcer disease or diabetes mellitus
8. Pregnant or Nursing
9. Medication exclusions included:
   oral, inhaled or nasal steroids for past 4 weeks
   Salmeterol for past 4 days
   Astemizole for past 12 weeks
   Nedocromil sodium, sodium cromoglycate for past 4 weeks
   Long acting methlyxanthines for past 2 days
   Short acting methylxanthines for past 12 hours
   Montelukast or zafirlukast for past 7 days

Enrollment in the Clinical Trial

Thirty nine subjects were eligible and enrolled in this clinical trial, and 37 subjects proceeded with the first bronchoscopy (two subjects withdrew in the run-in) although epithelial brushing samples were inadequate from 2 of these subjects. Thirty-four subjects completed the second bronchoscopy (three subjects dropped-out before the second bronchoscopy). Subjects dropping out before the second bronchoscopy comprised one subject who was withdrawn based on investigator judgment that paradoxical vocal cord movement during the first bronchoscopy could place the subject at increased risk at subsequent bronchoscopy, 1 subject who developed an asthma exacerbation between the first and second bronchoscopy, and 1 subject who had dark bronchoalveolar lavage fluid and was subsequently found to have an occupational exposure. One additional subject underwent the second bronchoscopy but did not have brushings due to coughing during the procedure. Of the 33 subjects with two bronchoscopies and brushings, one had too little RNA recovered on the first bronchoscopy for microarray analysis. Excluding this subject and the subject with the occupational exposure, 35 samples were available for microarray analysis from the first bronchoscopy and matched samples were available for 32 of these subjects at the second bronchoscopy. Baseline gene expression data from the three subjects with high quality RNA from only the first bronchoscopy are included in the analyses presented here. Of these 32 subjects, 29 subjects completed visit 4, 27 completed visit 5, and 26 completed visit 6. In summary, 2 subjects had asthma exacerbations (1 study procedure-related, 1 related to a URI), 2 had another study related adverse event (dysphonia, mood change), 2 were discharged from the study due to investigator concern before bronchoscopy, 1 was discharged for occupational exposure, 3 withdrew, and 1 subject was lost to follow-up.

ELISA

ELISA for serpinB2 protein levels in bronchoalveolar lavage fluid was performed using a commercially available kit (Imubind PAI-2 ELISA, American Diagnostica Inc, Stamford Conn.) according to the manufacturer's directions.

Immunohistochemistry

Paraffin-embedded sections were deparaffinized and re-hydrated to water. Next, all sections were incubated in 3% hydrogen peroxide/absolute methanol for 10 minutes to block endogenous peroxidase activity. Sections were then blocked for non-specific binding of antibodies by incubation in 1% goat serum for 30 minutes at 23° C. Sections were blotted of excess serum and incubated in primary antibody diluted in 1% goat serum/0.3% Tween-20/PBS (anti-OSF2 at 1:16,000 and anti-PN at 1:32,000) for 1 hour at 23° C. Sections were then incubated for 1 hour at 23° C. in secondary antibody, which was a biotinylated goat anti-rabbit antibody (Vector Labs; Burlingame, Calif., USA) for anti-OSF2 and anti-PN antibodies (1:500 dilution). Next, sections were incubated in ABC reagent (Vector Labs; Burlingame, Calif., USA) for 1 hour at 23° C., followed by DAB Plus reagent (Zymed Labs; South San Francisco, Calif., USA) for 10 minutes. Last, they were counterstained with Gill's #3 hematoxylin for 5 seconds, rinsed briefly in tap water, dehydrated, cleared, and mounted with a #1.5 coverslip using cytoseal.

Design-Based Stereology

For analysis of periostin expression, six endobronchial biopsies were obtained from 2^{nd}-through 5^{th}-order carinae contralateral to the site of bronchoalveolar lavage. Biopsies were formalin-fixed and paraffin-embedded and then analyzed using methods of tissue immunohistochemistry (described above) and design-based stereology.⁶ First, the volume of immunohistochemical staining, the volume of submucosal tissue surveyed, and the surface area of the epithelial basal lamina surveyed were determined by point and intersection counting using an integrated microscope (Olympus; Albertslund, Denmark), video camera (JVC Digital Color, JVC A/S; Tatstrup, Denmark), automated microscope stage, and computer (Dell Optiplex GS270 PC running Computer-Assisted Stereology Toolbox [C.A.S.T.] Software, Olympus Denmark; Albertslund, Denmark). A line segment grid was superimposed on systematically randomly selected microscopic fields. Points overlying submucosal tissue that is immunostained and unstained were counted separately along with intersections of test lines with basal lamina. The measurements were recorded by a blinded investigator using a 20× objective lens. The volume density of immunostaining was calculated by quantification of the volume of stained submucosa referenced to the surface area of the basal lamina surveyed (μm³/μm²).

Epithelial Cell Culture Methods

Human primary bronchial airway epithelial cells were purchased (Clonetics—Cambrex Bio Science, Walkersville Md.) and grown as described by Gray et al.⁷ Cells were purchased at passage 1 and expanded in T25 flasks until they reached 80% confluency at which time they were passaged onto 6.5 mm Transwell permeable supports at a density of 132,000 cells/filter. The cells were grown submerged in Gray's medium until approximately day 7 to 9 at which point an air-liquid interface was established. Cells became fully differentiated (ciliated and mucous producing) as determined by transepithelial voltage and resistance after 2-3 weeks on the filters. Cells were exposed to the recombinant human IL-13 (R&D Systems, Minneapolis Minn.) for up to four days at concentrations ranging from 0.1 ng/ml to 10 ng/ml. Filters were cut from the support and submerged in RLT lysis buffer (Qiagen Inc, Valencia Calif.) and stored at −80° C. before analysis of gene expression by real time RT-PCR.

### REFERENCES FOR SUPPLEMENT TO EXAMPLE 1

1. Kuperman D A, Lewis C C, Woodruff P G, et al. Dissecting asthma using focused transgenic modeling and functional genomics. J Allergy Clin Immunol 2005; 116(2):305-11.
2. Fahy J V, Wong H, Liu J, Boushey H A. Comparison of samples collected by sputum induction and bronchoscopy from asthmatic and healthy subjects. Am J Respir Crit. Care Med 1995; 152(1):53-8.
3. Woodruff P G, Dolganov G M, Ferrando R E, et al. Hyperplasia of smooth muscle in mild to moderate asthma without changes in cell size or gene expression. Am J Respir Crit. Care Med 2004; 169(9):1001-6.
4. Hays S R, Woodruff P G, Khashayar R, et al. Allergen challenge causes inflammation but not goblet cell degranulation in asthmatic subjects. J Allergy Clin Immunol 2001; 108(5):784-90.
5. Chai H, Farr R S, Froehlich L A, et al. Standardization of bronchial inhalation challenge procedures. J Allergy Clin Immunol 1975; 56(4):323-7.
6. Bolender R P, Hyde D M, Dehoff R T. Lung morphometry: a new generation of tools and experiments for organ, tissue, cell, and molecular biology. Am J Physiol 1993;265(6 Pt 1):L521-48.

7. Gray T E, Guzman K, Davis C W, Abdullah L H, Nettesheim P. Mucociliary differentiation of serially passaged normal human tracheobronchial epithelial cells. Am J Respir Cell Mol Biol 1996; 14(1):104-12.

TABLE 1s
Genes differentially expressed in epithelial brushings by microarray analysis
in subjects with asthma as compared to healthy control subjects.

| | Gene | | Entrez | Fold- | Log₂ | |
|---|---|---|---|---|---|---|
| Affymetrix ID | Symbol | Gene Name | Gene ID | difference | intensity | p-value |
| 210107_at | CLCA1 | chloride channel, calcium | 1179 | 6.2 | 4.9 | 0.033 |
| | | activated, family member 1 | | | | |
| 210809_s_at | POSTN | periostin, osteoblast specific factor | 10631 | 4.4 | 9.0 | 0.0015 |
| 204919_at | PRR4 | proline rich 4 (lacrimal) | 11272 | 3.9 | 6.3 | 0.049 |
| 204614_at | SERPINB2 | serine (or cysteine) proteinase | 5055 | 3.5 | 7.8 | 0.047 |
| | | inhibitor, clade B (ovalbumin), | | | | |
| | | member 2 | | | | |
| 205624_at | CPA3 | carboxypeptidase A3 (mast cell) | 1359 | 3.4 | 8.0 | 0.0069 |
| 207134_x_at | TPSB2 | tryptase beta 2 | 64499 | 2.2 | 7.1 | 0.0062 |
| 205683_x_at | TPSAB1 | tryptase alpha/beta 1 | 7177 | 2.1 | 7.1 | 0.022 |
| 1559584_a_at | C16orf54 | chromosome 16 open reading | 283897 | 1.4 | 11.3 | 0.015 |
| | | frame 54 | | | | |
| 201061_s_at | STOM | stomatin | 2040 | 1.3 | 11.6 | 0.043 |
| 224782_at | ZMAT2 | zinc finger, matrin type 2 | 153527 | 1.3 | 9.9 | 0.038 |
| 205002_at | DJ159A19.3 | hypothetical protein DJ159A19.3 | 27245 | 1.2 | 7.3 | 0.026 |
| 217313_at | — | — | — | 1.2 | 7.3 | 0.0074 |
| 1555256_at | EVC2 | Ellis van Creveld syndrome 2 | 132884 | 1.1 | 5.6 | 0.032 |
| | | (limbin) | | | | |
| 237690_at | GPR115 | G protein-coupled receptor 115 | 221393 | −1.2 | 4.2 | 0.019 |
| 212820_at | DMXL2 | Dmx-like 2 | 23312 | −1.2 | 10.6 | 0.0003 |
| 241774_at | — | — | — | −1.3 | 6.6 | 0.016 |
| 225987_at | TNFAIP9 | tumor necrosis factor, alpha- | 79689 | −1.7 | 11.3 | 0.0078 |
| | | induced protein 9 | | | | |
| 213432_at | MUC5B | mucin 5, subtype B, | 4587 | −1.9 | 5.5 | 0.016 |
| | | tracheobronchial | | | | |
| 241436_at | SCNN1G | sodium channel, nonvoltage-gated 1, | 6340 | −1.9 | 7.4 | 0.0027 |
| | | gamma | | | | |
| 219410_at | TMEM45A | transmembrane protein 45A | 55076 | −2.0 | 10.8 | 0.0006 |
| 1556185_a_at | — | Homo sapiens, clone | — | −2.0 | 6.4 | 0.0061 |
| | | IMAGE: 5260162, mRNA | | | | |
| 230378_at | SCGB3A1 | secretoglobin, family 3A, member 1 | 92304 | −2.1 | 13.3 | 0.0096 |

TABLE 2s
Genes differentially expressed in epithelial brushings by microarray
analysis in smokers as compared to healthy control subjects.

| | Gene | | Entrez | Fold- | Log₂ | |
|---|---|---|---|---|---|---|
| Affymetrix ID | Symbol | Gene name | Gene ID | difference | intensity | p-value |
| 206561_s_at | AKR1B10 | aldo-keto reductase family 1, | 57016 | 13.6 | 7.4 | <0.0001 |
| | | member B10 (aldose reductase) | | | | |
| 201387_s_at | UCHL1 | ubiquitin carboxyl-terminal esterase | 7345 | 10.9 | 6.5 | <0.0001 |
| | | L1 (ubiquitin thiolesterase) | | | | |
| 202437_s_at | CYP1B1 | cytochrome P450, family 1, | 1545 | 10.4 | 6.6 | 0.021 |
| | | subfamily B, polypeptide 1 | | | | |
| 224646_x_at | H19 | H19, imprinted maternally expressed | 283120 | 6.8 | 9.7 | 0.015 |
| | | untranslated mRNA | | | | |
| 205623_at | ALDH3A1 | aldehyde dehydrogenase 3 family, | 218 | 5.1 | 9.7 | 0.0069 |
| | | memberA1 | | | | |
| 202831_at | GPX2 | glutathione peroxidase 2 | 2877 | 4.9 | 7.5 | <0.0001 |
| | | (gastrointestinal) | | | | |
| 209921_at | SLC7A11 | solute carrier family 7, (cationic | 23657 | 4.6 | 6.7 | <0.0001 |
| | | amino acid transporter, y+ system) | | | | |
| | | member 11 | | | | |
| 224279_s_at | CABYR | calcium-binding tyrosine-(Y)- | 26256 | 4.3 | 7.5 | <0.0001 |
| | | phosphorylation regulated | | | | |
| | | (fibrousheathin 2) | | | | |
| 1555854_at | AKR1C2 | Aldo-keto reductase family 1, | 1646 | 3.9 | 8.6 | 0.0068 |
| | | member C2 (dihydrodiol | | | | |
| | | dehydrogenase 2; bile acid binding | | | | |
| | | protein; 3-alpha hydroxysteroid | | | | |
| | | dehydrogenase, type III) | | | | |
| 1569378_at | — | Hypothetical gene supported by | 399983 | 3.8 | 6.0 | 0.0026 |
| | | AK090616 | | | | |
| 209875_s_at | SPP1 | secreted phosphoprotein 1 | 6696 | 3.7 | 6.1 | 0.018 |
| | | (osteopontin, bone sialoprotein I, | | | | |
| | | early T-lymphocyte activation 1) | | | | |
| 204059_s_at | ME1 | malic enzyme 1, NADP(+)- | 4199 | 3.4 | 7.3 | 0.0001 |
| | | dependent, cytosolic | | | | |
| 1553602_at | — | — | — | 3.3 | 8.2 | 0.040 |
| 210505_at | ADH7 | alcohol dehydrogenase 7 (class IV), | 131 | 3.3 | 9.2 | 0.0004 |
| | | mu or sigma polypeptide | | | | |
| 216594_x_at | AKR1C1 | aldo-keto reductase family 1, | 1645 | 2.9 | 10.7 | 0.0001 |
| | | member C1 (dihydrodiol | | | | |
| | | dehydrogenase 1; 20-alpha (3-alpha)- | | | | |
| | | hydroxysteroid dehydrogenase) | | | | |
| 206515_at | CYP4F3 | cytochrome P450, family 4, | 4051 | 2.7 | 6.6 | 0.0002 |
| | | subfamily F, polypeptide 3 | | | | |
| 201468_s_at | NQO1 | NAD(P)H dehydrogenase, quinone 1 | 1728 | 2.6 | 10.7 | 0.0003 |
| 206153_at | CYP4F11 | cytochrome P450, family 4, | 57834 | 2.5 | 5.3 | 0.0002 |
| | | subfamily F, polypeptide 11 | | | | |
| 204532_x_at | UGT1A10 | UDP glycosyltransferase 1 family, | 54575 | 2.3 | 7.3 | 0.0001 |
| | | polypeptide A10 | | | | |
| 1559072_a_at | KIAA1904 | KIAA1904 protein | 114794 | 2.1 | 7.3 | 0.018 |
| 206254_at | EGF | epidermal growth factor (beta- | 1950 | 2.0 | 5.9 | 0.018 |
| | | urogastrone) | | | | |
| 237351_at | LOC284825 | Hypothetical protein LOC284825 | 284825 | 2.0 | 5.2 | 0.0015 |
| 233076_at | C10orf39 | chromosome 10 open reading frame 39 | 282973 | 1.9 | 5.1 | 0.0001 |
| 225609_at | GSR | glutathione reductase | 2936 | 1.8 | 11.0 | 0.0008 |
| 210769_at | CNGB1 | cyclic nucleotide gated channel beta 1 | 1258 | 1.6 | 7.0 | 0.044 |
| 202804_at | ABCC1 | ATP-binding cassette, sub-family C | 4363 | 1.6 | 11.4 | <0.0001 |
| | | (CFTR/MRP), member 1 | | | | |
| 230953_at | UGT1A6 | UDP glycosyltransferase 1 family, | 54578 | 1.5 | 5.0 | 0.037 |
| | | polypeptide A9 | | | | |
| 235529_x_at | SAMHD1 | SAM domain and HD domain 1 | 25939 | −1.5 | 11.4 | 0.032 |
| 209200_at | MEF2C | MADS box transcription enhancer | 4208 | −1.6 | 7.2 | 0.031 |
| | | factor 2, polypeptide C (myocyte | | | | |
| | | enhancer factor 2C) | | | | |
| 230433_at | LOC400764 | hypothetical gene supported by | 400764 | −1.6 | 7.5 | 0.0074 |
| | | AK094796 | | | | |
| 205632_s_at | PIP5K1B | phosphatidylinositol-4-phosphate 5- | 8395 | −1.6 | 7.3 | 0.048 |
| | | kinase, type I, beta | | | | |
| 227155_at | LMO4 | LIM domain only 4 | 8543 | −1.8 | 9.1 | 0.012 |
| 204041_at | MAOB | monoamine oxidase B | 4129 | −1.8 | 7.9 | 0.0015 |
| 227702_at | CYP4X1 | cytochrome P450, family 4, | 260293 | −2.1 | 8.0 | 0.0020 |
| | | subfamily X, polypeptide 1 | | | | |
| 204179_at | MB | myoglobin | 4151 | −2.2 | 8.8 | 0.0015 |
| 1553604_at | ABCA13 | ATP-binding cassette, sub-family A | 154664 | −2.3 | 10.2 | 0.0018 |
| | | (ABC1), member 13 | | | | |
| 236261_at | OSBPL6 | Oxysterol binding protein-like 6 | 114880 | −2.4 | 9.8 | 0.018 |
| 219410_at | TMEM45A | transmembrane protein 45A | 55076 | −2.8 | 10.7 | 0.0040 |
| 202746_at | ITM2A | integral membrane protein 2A | 9452 | −2.9 | 9.5 | 0.0090 |
| 214456_x_at | SAA1 | serum amyloid A1 | 6288 | −4.5 | 9.3 | 0.0033 |

TABLE 3s
Corticosteroid responsive genes identified by microarray analysis
in subjects in the randomized placebo-controlled trial.

| | Gene | | Entrez | Fold-difference | | | Log₂ |
|---|---|---|---|---|---|---|---|
| Affymetrix ID | Symbol | Gene name | Gene ID | Placebo | Flovent | Combined | intensity |
| 228854_at | — | Transcribed locus | — | −1.4 | 7.3 | 10.5 | 7.0 |
| 224840_at | FKBP51 | FK506 binding protein 51 | 2289 | −1.3 | 3.6 | 4.4 | 8.9 |
| 204457_s_at | GAS1 | growth arrest-specific 1 | 2619 | −1.2 | 2.7 | 3.3 | 7.1 |
| 227949_at | PHACTR3 | phosphatase and actin | 116154 | −1.0 | 3.1 | 3.2 | 6.3 |
| | | regulator 3 | | | | | |
| 219735_s_at | TFCP2L1 | transcription factor CP2- | 29842 | 1.0 | 3.1 | 3.0 | 8.1 |
| | | like 1 | | | | | |
| 207547_s_at | TU3A | TU3A protein | 11170 | −1.2 | 2.3 | 2.8 | 7.4 |
| 228858_at | — | Transcribed locus | — | −1.2 | 2.4 | 2.8 | 7.4 |
| 231262_at | — | Transcribed locus | — | −1.2 | 2.4 | 2.8 | 5.1 |
| 1555318_at | HIF3A | hypoxia inducible factor 3, | 64344 | −1.0 | 2.7 | 2.7 | 4.9 |
| | | alpha subunit | | | | | |
| 214414_x_at | HBA2 | hemoglobin, alpha 2 /// | 3040 | 1.1 | 3.0 | 2.7 | 7.6 |
| | | hemoglobin, alpha 2 | | | | | |
| 208763_s_at | TSC22D3 | TSC22 domain family 3 | 1831 | −1.3 | 2.0 | 2.5 | 7.5 |
| 203543_s_at | KLF9 | Kruppel-like factor 9 | 687 | −1.1 | 2.2 | 2.4 | 5.8 |
| 205220_at | GPR109B | G protein-coupled receptor | 8843 | −1.2 | 1.9 | 2.4 | 7.8 |
| | | 109B /// G protein-coupled | | | | | |
| | | receptor 109B | | | | | |
| 1563473_at | PPP1R16B | Protein phosphatase 1, | 26051 | −1.2 | 2.1 | 2.4 | 5.7 |
| | | regulatory (inhibitor) | | | | | |
| | | subunit 16B | | | | | |
| 244677_at | — | — | — | −1.3 | 1.8 | 2.3 | 6.4 |
| 210168_at | C6 | complement component 6 | 729 | −1.3 | 1.6 | 2.2 | 7.2 |
| 230782_at | — | CDNA FLJ33419 fis, | — | −1.1 | 2.1 | 2.2 | 7.4 |
| | | clone BRACE2019877 | | | | | |
| 236950_s_at | LOC157381 | hypothetical protein | 157381 | −1.3 | 1.7 | 2.1 | 7.1 |
| | | LOC157381 | | | | | |
| 205384_at | FXYD1 | FXYD domain containing | 5348 | −1.5 | 1.4 | 2.1 | 8.5 |
| | | ion transport regulator 1 | | | | | |
| | | (phospholemman) | | | | | |
| 229483_at | UBE2H | Ubiquitin-conjugating | 7328 | −1.1 | 2.0 | 2.1 | 7.3 |
| | | enzyme E2H (UBC8 | | | | | |
| | | homolog, yeast) | | | | | |
| 212992_at | C14orf78 | chromosome 14 open | 113146 | −1.1 | 1.8 | 2.1 | 8.1 |
| | | reading frame 78 | | | | | |
| 209116_x_at | HBB | hemoglobin, beta /// | 3043 | 1.1 | 2.5 | 2.1 | 8.6 |
| | | hemoglobin, beta | | | | | |
| 227061_at | — | CDNA FLJ44429 fis, | — | −1.3 | 1.6 | 2.0 | 8.2 |
| | | clone UTERU2015653 | | | | | |
| 213836_s_at | WIPI49 | WD40 repeat protein | 55062 | −1.1 | 1.7 | 2.0 | 8.7 |
| | | Interacting with | | | | | |
| | | phosphoInositides of | | | | | |
| | | 49 kDa | | | | | |
| 218687_s_at | MUC13 | mucin 13, epithelial | 56667 | 1.3 | −1.6 | −2.0 | 8.9 |
| | | transmembrane | | | | | |
| 210809_s_at | POSTN | periostin, osteoblast | 10631 | 1.0 | −2.1 | −2.1 | 10.4 |
| | | specific factor | | | | | |
| 206224_at | CST1 | cystatin SN | 1469 | −1.1 | −2.4 | −2.1 | 6.5 |
| 205890_s_at | UBD | ubiquitin D | 10537 | 1.1 | −2.0 | −2.1 | 9.0 |
| 210107_at | CLCA1 | chloride channel, calcium | 1179 | −1.1 | −2.7 | −2.4 | 7.2 |
| | | activated, family member 1 | | | | | |
| 204614_at | SERPINB2 | serine (or cysteine) | 5055 | 1.2 | −2.5 | −2.9 | 8.7 |
| | | proteinase inhibitor, clade B | | | | | |
| | | (ovalbumin), member 2 | | | | | |

TABLE 5s
Genes previously analyzed by PCR in a subset of these asthmatic
and healthy control subjects and published to support observations
made using mouse models of airway inflammation.¹

| Symbol | Description |
|---|---|
| Clca1 | Chloride channel calcium activated 1 |
| Itln1 | Intelectin 1 |
| Alox15 | 15-lipoxygenase |
| Tff2 | Trefoil factor 2 |
| Muc5ac | Mucin 5, subtypes A and C |
| Tff1 | Trefoil factor 1 |
| Agr2 | Anterior gradient 2 |
| Slc26a4 | Solute carrier family 26, member 4 |
| Scin | Scinderin |
| Muc5b | Mucin 5, subtype B |
| AMCase | Acidic mammalian chitinase |

TABLE 4s
SerpinB2 protein levels in bronchoalveolar lavage increased
in all subjects at the follow-up bronchoscopy

| | | Mean | | |
|---|---|---|---|---|
| Group | Bronchoscopy | (ng/ml) | SD | |
| Placebo | Pre-placebo | 0.40 | 0.95 | |
| | Post-placebo | 3.43 | 4.68 | p = 0.04 as compared |
| | | | | to pre-placebo |
| Fluticasone | Pre-fluticasone | 0.16 | 0.29 | |
| | Post-fluticasone | 0.91 | 1.08 | p = 0.03 as compared |
| | | | | to pre-fluticasone |

### Example 2

Airway epithelial cells are stimulated with periostin protein to determine the effect on epithelial cell gene expression. Epithelial cell responses to periostin include upregulation of anti-apoptosis pathways, upregulation of fibrosis- or vascular-related genes, or changes in mucin genes.

### Example 3

Periostin is overexpressed in airway epithelial cells and the over-expressing cells are used in co-culture experiments and the effects of periostin on airway smooth muscle proliferation and vascular cell proliferation (HUVECs) are determined.

### Example 4

Epithelial Mucin Stores are increased in the Large Airways of Smokers with Airflow Obstruction. The pathophysiology of increased epithelial mucins in smokers is uncertain, as is the relationship between mucin pathology and obstruction to airflow.

We collected bronchial biopsies and epithelial brushings from 24 smokers with and without airflow obstruction and 19 non-smoking healthy control subjects. Epithelial mucin stores, mucin immunostains, and goblet cell morphology were quantified in bronchial biopsies using stereology, and mucin gene expression was quantified in epithelial brushings using real-time RT-PCR.

Goblet cell size and number were higher than normal in smokers (both p<0.05) leading to a 2.2-fold increase in stored mucin in the epithelium (p=0.001). The increase in stored mucin occurred because of an increase in MUC5AC (p=0.018), and despite a decrease in MUC5B (p<0.0001). Stored mucin was significantly higher in the subgroup of smokers with airflow obstruction (p=0.029).

Epithelial mucin stores are increased in habitual smokers because of goblet cell hypertrophy and hyperplasia, and the pattern of mucin gene expression is abnormal. The highest epithelial mucin stores are found in smokers with airflow obstruction, suggesting a mechanistic link between epithelial mucin dysregulation and airflow obstruction. Normalizing epithelial mucins may improve airflow in habitual smokers.

### Example 5

In further analysis of the same dataset we have identified six genes (Table 4) which are differentially expressed in airway epithelial brushings from asthmatics asthma or altered by corticosteroid treatment of asthmatic subjects and which have not previously been implicated in asthma or in the mechanism of steroid benefit in asthma.

TABLE 4
Additional five genes of interest from the dataset

| 1 | 2 | 3 | 4 |
|---|---|---|---|
| | Asthma/Healthy ratio PLACEBO Change | | |
| | (A1FLUTICASONE Change ( [symbol] asthma = 43; | | |
| | healthy = AVERAGE (n = 15) AVERAGE (n = 16) | | |
| CST1 | 50.73 | 1.52 | 5.97 |
| CST4 | 52.42 | 1.60 | 5.41 |
| SERPINB1 [symbol] | 3.71 | 1.00 | 2.31 |
| HDAC9 | 1.61 | 0.80 | 2.10 |
| CEACAM5 | 3.08 | 1.03 | 1.88 |

Fluticasone is an inhaled corticosteriod. Column 4 shows the ratio of gene expression in a [symbol] epithelial brushing cells before and after treatment for one week. A number greater than o [symbol] indicates suppression of gene expression by steroids.

We also examined the relationship between baseline gene expression and change in FEV1 in response to treatment with fluticasone for 4 or 8 weeks. We found that most of the six genes shows a relationship with baseline expression (Table 5). Furthermore, when we examined the relationship between the change in gene expression induced by one week of treatment with fluticasone and the changes in FEV1 seen at 4 and 8 weeks of fluticasonetreatment we also found relationships for CEACAM5 and Serpin B10.

TABLE 5
Relationship between baseline gene expression and change in FEV1
in response to treatment with fluticasone for 4 or 8 weeks

| | | 4 weeks | | 8 weeks |
|---|---|---|---|---|
| | r | p | r | p |
| cst1 | 0.582 | 0.014 | 0.519 | 0.039 |
| cst4 | 0.573 | 0.016 | 0.523 | 0.038 |
| ceacam | 0.629 | 0.007 | 0.393 | 0.132 |
| serpB10 | 0.601 | 0.011 | 0.455 | 0.076 |
| hdac9 | 0.156 | 0.549 | 0.138 | 0.611 |
| fkbp51 | −0.628 | 0.007 | −0.630 | 0.009 |

TABLE 6
Relationship between the change in gene expression induced
by one week of treatment with fluticasone and the changes
in FEV1 seen at 4 and 8 weeks of fluticasone treatment

| | | 4 weeks | | 8 weeks |
|---|---|---|---|---|
| | r | p | r | P |
| cst1 | −0.012 | 0.966 | −0.388 | 0.153 |
| cst4 | −0.020 | 0.943 | −0.429 | 0.111 |
| ceacam | −0.476 | 0.063 | −0.353 | 0.197 |
| serpB10 | −0.686 | 0.003 | −0.617 | 0.014 |
| hdac9 | 0.055 | 0.839 | 0.472 | 0.076 |
| fkbp51 | −0.095 | 0.726 | 0.389 | 0.152 |

### REFERENCES

1. Malerba G, Pignatti P F. A review of asthma genetics: gene expression studies and recent candidates. J Appl Genet. 2005; 46(1):93-104.
2. Donohue J F, Ohar J A. Effects of corticosteroids on lung function in asthma and chronic obstructive pulmonary disease. Proc Am Thorac Soc 2004; 1(3):152-60.
3. Doherty D E. The pathophysiology of airway dysfunction. Am J Med 2004;117 Suppl 12A:11S-23S.
4. Kuperman D A, Lewis C C, Woodruff P G, et al. Dissecting asthma using focused transgenic modeling and functional genomics. J Allergy Clin Immunol 2005;116(2):305-11.
5. Gentleman R C, Carey V J, Bates D M, et al. Bioconductor: open software development for computational biology and bioinformatics. Genome Biol 2004; 5(10):R80.
6. Bolstad B M, Irizarry R A, Astrand M, Speed T P. A comparison of normalization methods for high density oligonucleotide array data based on variance and bias. Bioinformatics 2003; 19(2):185-93.
7. Irizarry R A, Bolstad B M, Collin F, Cope L M, Hobbs B, Speed T P. Summaries of Affymetrix GeneChip probe level data. Nucleic Acids Res 2003; 31(4):e15.
8. Nakanishi A, Morita S, Iwashita H, et al. Role of gob-5 in mucus overproduction and airway hyperresponsiveness in asthma. Proc Natl Acad Sci USA 2001; 98(9):5175-80.
9. Zhou Y, Dong Q, Louahed J, et al. Characterization of a calcium-activated chloride channel as a shared target of Th2 cytokine pathways and its potential involvement in asthma. Am J Respir Cell Mol Biol 2001; 25(4):486-91.
10. Toda M, Tulic M K, Levitt R C, Hamid Q. A calcium-activated chloride channel (HCLCA1) is strongly related to IL-9 expression and mucus production in bronchial epithelium of patients with asthma. J Allergy Clin Immunol 2002; 109(2):246-50.
11. Hoshino M, Morita S, Iwashita H, et al. Increased expression of the human Ca2+-activated C1-channel 1 (CaCC1) gene in the asthmatic airway. Am J Respir Crit. Care Med 2002; 165(8):1132-6.
12. Davies T H, Ning Y-M, Sanchez E R. A New First Step in Activation of Steroid Receptors. Hormone-induced switching of fkbp51 and fkbp52 immunophilins. J Biol Chem 2002; 277(7):4597-600.
13. Boyce J A. The role of mast cells in asthma. Prostaglandins Leukot Essent Fatty Acids 2003;69(2-3): 195-205.
14. Marone G, Triggiani M, Genovese A, Paulis A D. Role of human mast cells and basophils in bronchial asthma. Adv Immunol 2005; 88:97-160.
15. Gillan L, Matei D, Fishman D A, Gerbin C S, Karlan B Y, Chang D D. Periostin secreted by epithelial ovarian carcinoma is a ligand for alpha(V) beta(3) and alpha(V) beta(5) integrins and promotes cell motility. Cancer Res 2002; 62(18):5358-64.
16. Katsuragi N, Morishita R, Nakamura N, et al. Periostin as a Novel Factor Responsible for Ventricular Dilation. Circulation 2004; 110(13):1806-13.
17. Shao R, Bao S, Bai X, et al. Acquired expression of periostin by human breast cancers promotes tumor angiogenesis through up-regulation of vascular endothelial growth factor receptor 2 expression. Mol Cell Biol 2004; 24(9):3992-4003.
18. Bao S, Ouyang G, Bai X, et al. Periostin potently promotes metastatic growth of colon cancer by augmenting cell survival via the Akt/PKB pathway. Cancer Cell 2004; 5(4):329-39.
19. Grunig G, Warnock M, Wakil A E, et al. Requirement for IL-13 independently of IL-4 in experimental asthma. Science 1998; 282(5397):2261-3.
20. Wills-Karp M, Luyimbazi J, Xu X, et al. Interleukin-13: central mediator of allergic asthma. Science 1998; 282(5397):2258-61.
21. Medcalf R L, Stasinopoulos S J. The undecided serpin. The ins and outs of plasminogen activator inhibitor type 2. Febs J 2005; 272(19):4858-67.
22. Cohn L, Elias J A, Chupp G L. Asthma: mechanisms of disease persistence and progression. Annu Rev Immunol 2004; 22:789-815.
23. Rogatsky I, Wang J C, Derynck M K, et al. Target-specific utilization of transcriptional regulatory surfaces by the glucocorticoid receptor. Proc Natl Acad Sci USA 2003; 100(24):13845-50.
24. Vermeer H, Hendriks-Stegeman B I, van der Burg B, van Buul-Offers S C, Jansen M. Glucocorticoid-Induced Increase in Lymphocytic FKBP51 Messenger Ribonucleic Acid Expression: A Potential Marker for Glucocorticoid Sensitivity, Potency, and Bioavailability. J Clin Endocrinol Metab 2003; 88(1):277-84.
25. Vermeer H, Hendriks-Stegeman B I, Suylekom D v, Rijkers G T, Buul-Offers S Cv, Jansen M. An in vitro bioassay to determine individual sensitivity to glucocorticoids: induction of FKBP51 mRNA in peripheral blood mononuclear cells. Molecular and Cellular Endocrinology 2004;218(1-2):49-55.
26. Vermeer H, Hendriks-Stegeman B I, Verrijn Stuart A A, van Buul-Offers S C, Jansen M. A comparison of in vitro bioassays to determine cellular glucocorticoid sensitivity. Eur J Endocrinol 2004; 150(1):41-7.
27. Reynolds P D, Ruan Y, Smith D F, Scammell J G. Glucocorticoid Resistance in the Squirrel Monkey Is Associated with Overexpression of the Immunophilin FKBP51. J Clin Endocrinol Metab 1999; 84(2):663-9.
28. Pauwels R A, Buist A S, Calverley P M, Jenkins C R, Hurd S S. Global strategy for the diagnosis, management, and prevention of chronic obstructive pulmonary disease. NHLBI/WHO Global Initiative for Chronic Obstructive Lung Disease (GOLD) Workshop summary. Am J Respir Crit Care Med 2001; 163(5):1256-76.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including the Background, Summary, Detailed Description, Examples, References, and Supplementary Appendix and Sequences listing section are hereby incorporated by reference in their entirety for all purposes and with particularity with the subject matter of the paragraph or section in which they are mentions. A mention of a reference or publication herein is not an admission that such is prior art.

What is claimed is:

1. A method of identifying agents useful in the treatment or prevention of asthma, allergic rhinitis, chronic obstructive pulmonary disease, or inflammatory or fibrotic diseases of the lung in a subject, said method comprising determining the ability of the agent modulate to CST1, HDAC9 or PRR4 levels or activity.

2. The method of claim 1, wherein the CST1, HDAC9 or PRR4 has a substantial sequence identity to a corresponding reference nucleic acid or polypeptide sequence disclosed herein.

3. The method of claim 2, wherein the sequence identity is at least 80%.

4. The method of claim 2, wherein the sequence identity is at least 95%.

5. The method of claim 1, wherein asthma is treated.

6. The method of claim 1, wherein the ability is determined by measuring the CST1, HDAC9 or PRR4 levels or activity in an experimental sample contacted with the agent and in a reference or control sample which was not contacted with the agent; and comparing the measurements of the experimental and reference samples.

7. The method of claim 6, wherein the tissue is airway epithelium.

8. The method of claim 7, wherein the airway epithelium is obtained from an asthmatic individual.

9. The method of claim 6, wherein the agent is siRNA which is capable of reducing the expression of CST1, HDAC9 or PRR4.

10. The method of claim 6, wherein the level of a polynucleotide which encodes CST1, HDAC9 or PRR4 is determined.

11. The method of claim 6, wherein the level of CST1, HDAC9 or PRR4 protein in a cell is measured.

12. The method of claim 6, wherein the ability of the agent to modulate CST1, HDAC9 or PRR4 activity is determined.

13. A method of identifying an individual having an increased susceptibility or resistance to asthma, said method comprising determining the levels or activity of CST1, HDAC9 or PRR4 in a tissue sample from the individual.

14. The method of claim 13, wherein the tissue sample comprises epithelial cells.

15. The method of claim 14, wherein the tissue sample is from the airway bronchi, or lung.

16. A method of diagnosing asthma, allergic rhinitis, chronic obstructive pulmonary disease, or inflammatory or fibrotic diseases of the lung in a subject, the method comprising the steps of:
(a) contacting a sample from the subject with a reagent that specifically binds to CST1, HDAC9 or PRR4 protein or nucleic acid; and
(b) determining the level of CST1, HDAC9 or PRR4 protein or nucleic acid expression in the sample as compared to a control sample, thereby diagnosing asthma, allergic rhinitis, chronic obstructive pulmonary disease, or inflammatory or fibrotic diseases of the lung in a subject.

17. A method of treating asthma, allergic rhinitis, chronic obstructive pulmonary disease, or inflammatory or fibrotic diseases of the lung in a subject, the method comprising the step of administering to a subject an anti-rejection drug that modulates PRR4 or HDAC9.

18. The method of claim 17, wherein the anti-rejection drug is formulated for administration by inhalation.
